(19) United States
(12) Patent Application Publication
(10) Pub. No.: US 2009/0005336 A1
(43) Pub. Date: Jan. 1, 2009

(54) Use of the microRNA miR-1 for the treatment, prevention, and diagnosis of cardiac conditions

(76) Inventor: Zhiguo Wang, Montreal (CA)

Correspondence Address: Louis Tessier, C.P. 54029, Mount-Royal, QC, H3P 3H4, CA

(21) Appl. No.: 12/149,803

(22) Filed: May 8, 2008

Related U.S. Application Data
(60) Provisional application No. 60/924,288, filed on May 8, 2007.

(51) Int. Cl. A61K 31/7105; C12Q 1/68; C07H 21/02

(52) U.S. Cl. 514/44; 435/6; 536/23.1

(57) ABSTRACT

Among >300 miRNAs known to date, miR-1 is considered muscle-specific. Here we show that that miR-1 overexpressed in individuals with coronary artery disease, and when overexpressed, it exacerbated arrhythmogenesis in both infarcted and normal hearts of rats whereas elimination of miR-1 by its antisense inhibitor relieved it. MiR-1 rendered slowed conduction and depolarized membrane by post-transcriptionally repressing KCNJ2 and GJA1 genes, likely accounting for its arrhythmogenic potential. Thus, miR-1 may have important pathophysiological functions in heart, being a novel antiarrhythmic target useful in the treatment and prevention of various cardiac pathologies.

This application claims priority from U.S. Provisional Patent Application Ser. No. 60/924,288 filed May 8, 2007, which is hereby incorporated by reference in its entirety.

### FIELD OF THE INVENTION

The present invention relates generally to the field of molecular biology. More particularly, it concerns methods and compositions involving microRNA (miRNAs) molecules. In addition, there are applications for miRNAs in diagnostics, therapeutics, and prognostics. Particularly, the present invention relates methods of preventing or treating a cardiac condition in a mammal by administering a therapeutically effective amount of an miR-1 inhibitor.

### BACKGROUND OF THE INVENTION

MicroRNAs (miRNAs) are endogenous relatively small noncoding RNAs that mediate posttranscriptional gene silencing by annealing to inexactly complementary sequences in the 3′UTRs of target mRNAs (1-3). mRNAs are an abundant RNA species both in terms of the sheer number of miRNAs in the genome (>1% of the predicted human genes) and in terms of their expression levels (some miRNAs >1000 copies per cell). However, in spite of the current ability to identify miRNAs, regulatory targets have not been well established and the function of miRNAs is poorly understood. The current understanding of the functions of miRNAs primarily relies on their tissue-specific or developmental stage-dependent expression patterns as well as their evolutionary conservation and thus is limited to developmental regulation and oncogenesis (2).

MiR-1 has been implicated in determination of the differentiated state and in myogenesis (5,6). Increasing expression of miR-1 was found in neonatal hearts, and substantially higher levels are maintained in adult hearts (4-6), indicating that it may have other cellular and pathophysiological functions in addition to myogenesis.

Accordingly, there is a need in the industry to utilize microRNAs for the treatment and prevention of disorders that are responsive to exposure to microRNAs.

The present description refers to a number of documents, the content of which is herein incorporated by reference in their entirety.

### SUMMARY OF THE INVENTION

In a broad aspect, the invention provides a method of preventing or treating a cardiac condition in a mammal, such as a human, the method comprising administering a therapeutically effective amount of an inhibitor of miR-1 to the mammal. For example, and non-limitingly, the cardiac condition is selected from the group consisting of cardiac arrhythmia, myocardial infarction, and coronary artery disease. Examples of cardiac arrhythmia include a ventricular arrhythmia, a widening of the QRS complex and a prolonged QT interval or a phase II arrhythmia.

In some embodiments of the invention, the inhibitor of miR-1 is a 2′-O-methyl-modified antisense oligoribonucleotide (AMO) specific to miR-1, for example a miR-1 having the nucleic acid sequence set forth in SEQ ID No: 1. In some embodiments, the inhibitor of miR-1 is the exact antisense of the mature miR-1 mRNA sequence. In some embodiments of the invention, the AMO has the nucleic acid sequence set forth in SEQ ID NO: 13.

The inhibitor is delivered prior to the onset of the cardiac condition or the inhibitor is delivered after the onset of the cardiac condition.

In another broad aspect, the invention provides a method of diagnosing a cardiac condition in a mammal, for example a human, the method comprising measuring the expression level of miR-1 in the mammal and comparing the expression level to a standard miR-1 level, wherein an increase in miR-1 expression level compared to the standard level in the mammal indicates the mammal has a cardiac condition.

For example, an increase of approximately 2.8-fold or more of miR-1 level indicates that the mammal has a cardiac condition.

In some embodiments of the invention, measuring the expression level of miR-1 comprises performing a reverse transcription polymerase chain reaction of miR-1 using a total RNA sample from the mammal.

Examples of cardiac conditions include arrhythmia, myocardial infarction, and coronary artery disease. Example of cardiac arrhythmia include a ventricular arrhythmia, a widening of the QRS complex and a prolonged QT interval or a phase II arrhythmia.

For example, the miR-1 has the nucleic acid sequence set forth in SEQ ID NO: 1.

The method of diagnosing is performed prior to the onset of any overt symptoms of the cardiac conditions occurring or the method of diagnosing is performed after the onset of any overt symptoms of the cardiac condition occurring.

In another broad aspect, the invention provides a method of inducing a cardiac condition in a mammal, for example a rat, the method comprising administering miR-1 to the cardiovascular system of the mammal in an amount effective to induce a cardiac condition. In some embodiments of the invention, the miR-1 has the nucleic acid sequence set forth in SEQ ID NO.: 2.

In another broad aspect, the invention provides an isolated nucleic acid, said nucleic acid having the sequence set forth in SEQ ID NOS:12 or 13.

In another broad aspect, the invention provides a method of preventing or treating a cardiac condition in a mammal, the method comprising administering a therapeutically effective amount of a compound that increases the expression of GJAI and KCNJ2.

Other objects, advantages and features of the present invention will become more apparent upon reading of the following non-restrictive description of preferred embodiments thereof, given by way of example only and in relation with the following Figures.

### BRIEF DESCRIPTION OF THE DRAWINGS

In the appended drawings:

FIG. 1 illustrates that miR-1 is arrhythmogenic/proarrhythmic in ischemic and normal hearts. Panel (a) illustrates increases in miR-1 level, determined by the mirVana™ qRT-PCR miRNA Detection Kit (Ambion) in conjunction with real-time PCR with SYBR Green I, with total RNA samples isolated from individuals suffered from coronary artery disease (CAD) and from male Wister rats of experimental myocardial infarction (MI) 12 h after occlusion of the left anterior descending artery. Comparisons were made between control non-ischemic hearts (Ctl) and MI; NIZ, non-ischemic zone; BZ, boarder zone; IZ, ischemic zone. Data are expressed as mean±SE normalized to Ctl and the number of RNA samples studied is indicated in the bracket. *p<0.05 vs. Ctl; +p<0.05 vs. IZ; unpaired Student t-test. Panel (b) illustrates examples of spontaneous arrhythmias in MI hearts, consisting of ventricular premature beat (VPB), ventricular tachycardia (VT) and ventricular fibrillation (VF), recorded with standard lead II ECG for a continuous period of 1 h starting from 12 h after MI, and miR-1-induced arrhythmias in time-matched non-ischemic healthy hearts (HH). SR: sinus rhythm in MI hearts; Ctl: sinus rhythm without miR-1 application in HH hearts. Delivery of AMO-1 (the antisense inhibitor oligonucleotides targeting miR-1, see Supplementary Methods for the sequence) suppressed arrhythmias. Panels (c) and (d) illustrate that miR-1 promotes ischemic arrhythmias in MI hearts (n=25) and induces arrhythmias in HH (n=30). In vivo gene transfer was performed to deliver wild-type miR-1 (WT miR-1, 50 mg in 100 ml), AMO-1 (80 mg in 100 ml), or mutant miR-1 (MT miR-1, 50 mg in 100 ml, bearing base substitutions at eight positions from the 5′ end of WT miR-1) to ventricular myocytes by direct intramuscular injection. Shown are data expressed as % of incidence (mean±SE) and the actual incidence is indicated by the numbers above each individual bar (same below). *p<0.05 vs. MI alone or HH Ctl; +p<0.05 vs. WT miR-1; c2-test. Panel (e) illustrates the effects of miR-1 on arrhythmias expressed as the Arrhythmia Score (AS) according to Curtis and Walker23. *p<0.05 vs. MI alone or Ctl; +p<0.05 vs. WT miR-1; unpaired Student t-test. Panel (f) illustrates epicardial conduction velocity (mean±SE) measured in isolated Langendorff-perfused hearts of MI (n=12) or HH (n=10) rats. The constructs were delivered before coronary artery occlusion by intramuscular injection. *p<0.05 vs. MI alone or Ctl; +p<0.05 vs. WT miR-1; unpaired Student t-test. Panel (g) illustrates the resting membrane potential (mean±SE) measured by standard microelectrode techniques in tissue strips dissected from ischemic zone and boarder zone (BZ) of MI hearts (n=9) and HH (n=8) 12 h following intramuscular injection delivery of varying constructs as described above. *p<0.05 AMO-1 vs. MI alone or Ctl; +p<0.05 WT miR-1 plus AMO-1 vs. WT miR-1 alone; unpaired Student t-test. (h) Changes of miR-1 level in rat myocardium, determined with the qRT-PCR miRNA Detection Kit (Ambion), with in vivo transfer of WT miR-1, AMO-1 or MT miR-1. *p<0.05; unpaired Student t-test. (see Supplementary Methods for detailed information on all aspects of the study).

FIG. 2 illustrates that miR-1 silences GJA1 (encoding connexin 43, Cx43 gap junction protein) and KCNJ2 (encoding Kir2.1, a major subunit of cardiac IK1) genes by repressing their translation into proteins. Panels (a) & (b) illustrate the effects of miR-1 on expression of GJA1 and KCNJ2 at the protein levels (Cx43 and Kir2.1, respectively) determined by Western blot with membrane samples extracted from healthy rat hearts (HH) or rats with myocardial infarction (MI). Upper sub-panels show examples of Western blot bands and lower sub-panels show mean±SE (n=12 MI hearts and n=10 HH hearts). Measurements were made 12 h after MI. Both Cx43 and Kir2.1 were reduced in MI and the reduction was exacerbated by miR-1, but alleviated by AMO-1. NIZ, non-ischemic zone; IZ, ischemic zone. *p<0.05; unpaired Student t-test. Panel (c) illustrates the verification of repression of Cx43 by miR-1 using immunohistochemical analysis. Cx43 is normally distributed at the intercalated discs and in MI, Cx43 is largely diminished and relocated to lateral surface membrane. AMO-1 rescued Cx43 presumably by inhibiting endogenous miR-1 that overexpresses in MI, whereas exogenous miR-1 exacerbates Cx43 downregulation, which is also reversed by AMO-1. AP: antigenic peptide. Panel (d) illustrates the verification of repression of Kir2.1 by miR-1 using whole-cell patch-clamp recording of IK1 in enzymatically isolated ventricular myocytes of rats. The raw traces shown were recorded by a 200-ms hyperpolarizing pulse to −120 mV from a holding potential of −20 mV. I-V curves are mean data from at least 8 cells for each condition. The data demonstrate that IK1 density is decreased in MI, which is rescued by AMO-1 presumably by inhibiting endogenous miR-1 that overexpresses in MI, whilst exogenous miR-1 exacerbates IK1 downregulation, which is reversed by AMO-1. For clarity, the outward IK1 at voltages between −80 mV and 0 mV is shown in the lower right sub-panel. Panel (e) illustrates the lack of significant effects of miR-1 on GJA1 and KCNJ2 mRNA levels, quantified by real-time RT-PCR, in both MI and HH rats. The RNA samples were extracted from the same hearts as for protein extraction described in panels (a) and (b). Ctl: control without miR-1 treatment. Panel (f) illustrates the verification of interactions between rat miR-1 (100 nM) and the 3′UTRs of rat GJA1 and KCNJ2 genes in HEK293 cells, determined by luciferase reporter activity. Cells (1×105/well) were transfected with 1 mg of varying constructs with lipofectamine 2000 (Supplementary Methods). MT miR-1 (100 nM) failed to affect luciferase activity with WT 3′UTR of GJA1 or KCNJ2, but it elicited similar repressing effects on luciferase reporter gene as WT miR-1 did when the 3′UTRs were also mutated to match the MT miR-1 sequence. AMO-1 concentration used was 100 nM. For rat genes: Mean±SE, n=8 batches of cells for each group; *p<0.05 vs. Ctl; +p<0.05 vs. WT miR-1; unpaired Student t-test. Panel (g) illustrates the verification of the 3′UTRs of human GJA1 and KCNJ2 genes as targets for human miR-1 in HEK293 cells. The lipofectamine 2000-mediated transfection procedures were the same as for panel (f). Quantitatively, the same effects were observed with human miR-1 and genes as with the rat constructs. Mean±SE, n=8 batches of cells for each group; *p<0.05 vs. Ctl; +p<0.05 vs. WT miR-1; unpaired Student t-test.

FIG. 3 illustrates the effects of miR-1 on the protein levels of Cx43 (in panel (a)) and Kir2.1 (in panel (b)) with membrane samples isolated from cultured neonatal rat ventricular myocytes24, determined by Western blot analysis. Cells were transfected with miR-1 alone or together with AMO-1 or mismatched AMO-1 (Mis-AMO-1), with lipofectamine 2000. AMO-1 reversed the repressing effects of miR-1 on Cx43 and Kir2.1, but Mis-AMO-1 failed to do so. Shown are mean±SE from 5 batches of cells. *p<0.05 vs. Ctl, +p<0.05 vs. miR-1 alone; unpaired Student t-test.

FIG. 4 illustrates the effects of GJA1 and KCNJ2 knockdown by specific siRNAs on arrhythmias. Panels (a) and (b) illustrate the proarrhythmic effects of the siRNAs targeting GJA1 and KCNJ2 in ischemic rat hearts (MI, n=11) and control healthy rat hearts (HH, n=15). In vivo gene transfer was performed to deliver the mixed siRNAs (Cx43-siRNA+Kir2.1-siRNA, 80 mg/each in 100 ml) or the mixed negative control siRNAs (Neg siRNA), together with the miR-1-specific antisense inhibitor oligonucleotides (AMO-1, 80 mg in 100 ml) to ventricular myocytes, by multiple-site intramuscular injection of liposome-treated constructs into the myocardium. The injections were made before coronary artery occlusion to establish MI within the area equivalent to infracted zone (left ventricular front wall proximal to the apex). Shown are data expressed as % of incidence (mean±SE) and the actual incidence is indicated by the numbers above each individual bar. *p<0.05 vs. MI alone or HH Ctl; +p<0.05 vs. siRNAs; c2-test. Panel (c) illustrates the effects of the siRNAs on expression of GJA1 and KCNJ2 at the protein level (Cx43 and Kir2.1, respectively) determined by Western blot with membrane samples extracted from healthy rat hearts subjected to in vivo transfection of varying constructs. Left sub-panel shows examples of Western blot bands and right panel shows mean±SE (n=5 independent samples for each group). Note that the effects of the siRNAs on arrhythmias and Cx43 and Kir2.1 protein levels were qualitatively the same as those of miR-1. *p<0.05 vs. Ctl; unpaired Student t-test. Labels for the bars are the same below. Panel (d) illustrates the effects of the siRNAs on expression of GJA1 and KCNJ2 at the mRNA levels determined by real-time RT-PCR with the total RNA samples extracted from healthy rat hearts subjected to in vivo transfection of varying constructs. In opposition to miR-1, the mixed siRNAs significantly downregulated GJA1 and KCNJ2 mRNA levels. Data are mean±SE (n=5 independent samples for each group); *p<0.05 vs. Ctl; unpaired Student t-test. Panel (e) illustrates the selective knockdown of miR-1 by co-injected AMO-1. Data are mean±SE (n=5 independent samples for each group); *p<0.05; unpaired Student t-test. This data demonstrate that direct knockdown of Cx43 and Kir2.1 by the siRNAs even when miR-1 levels had been reduced by AMO-1 produced similar effects on ischemic arrhythmogenesis as overexpression of miR-1, indicating that depression of Cx43 and Kir2.1 is a key link for the proarrhythmic action of miR-1.

FIG. 5 illustrates the complementary sequences between miR-1 and its putative sites within the 3′UTRs of human (H), rat (R), and mouse (M) GJA1 and KCNJ2 mRNAs, predicted with computational and bioinformatics-based approach using TargetScan hosted by Wellcome Trust Sanger Institute11. Watson-Crick complementarity is shown in bold and connected by “I”; the Genbank accession numbers are provided in the brackets. Note that there are two putative miR-1 target sites in the 3′UTR of GJA1. SEQ ID NOS:1, 2, and 3 refer to the human (AC103987), rat (DQ066650), and mouse (AJ459703) miR-1 sequences, respectively. SEQ ID NO:4 refers to the putative miR-1 site within the 3′UTRs of human (NM_{—}000165) mRNA. SEQ ID NOS:5 and 6 refer to the putative miR-1 site within the 3′UTRs of rat (BC081842) mRNA. SEQ ID NOS:7 and 8 refer to the putative miR-1 site within the 3′UTRs of mouse (BC006894) mRNAs. SEQ ID NOS:9, 10, and 11 refer to the putative miR-1 site within the 3′UTRs of human (AF153818), rat (NW_{—}047343), and mouse (NM_{—}008425) mRNAs, respectively. See also FIG. 12.

FIG. 6 illustrates the comparison of connexin43 (Cx43) and Kir2.1 protein levels between membrane samples isolated from individuals with healthy control hearts (HH) and from individuals suffered from coronary artery diseases (CAD), determined by Western blot analysis. The polyclonal antibodies to Cx43 and Kir2.1, respectively, were obtained from Santa Cruz (same below). Shown are mean±SE from 6 Ctl and 7 CAD; *p<0.05 vs. Ctl.; unpaired Student t-test.

FIG. 7 illustrates effects of miR-1 on the protein levels of Cx43 in panel (a) and Kir2.1 in panel (b) with membrane samples isolated from cultured neonatal rat ventricular myocytes³⁹, determined by Western blot analysis. Cells were transfected with miR-1 alone or together with AMO-1 or mismatched AMO-1 (Mis-AMO-1), with lipofectamine 2000 (Invitrogen). AMO-1 reversed the repressing effects of miR-1 on Cx43 and Kir2.1, but Mis-AMO-1 failed to do so. Shown are mean±SE from 5 batches of cells. *p<0.05 vs. Ctl, +p<0.05 vs. miR-1 alone; unpaired Student t-test.

FIG. 8 illustrates effects of GJA1 and KCNJ2 knockdown by specific siRNAs on arrhythmias. Panel (a) and (b) illustrate proarrhythmic effects of the siRNAs targeting GJA1 and KCNJ2 in ischemic rat hearts (MI, n=11) and control healthy rat hearts (HH, n=15). In vivo gene transfer was performed to deliver the mixed siRNAs (Cx43-siRNA+Kir2.1-siRNA, 80 μg/each in 100 μl) or the mixed negative control siRNAs (Neg siRNA), together with the miR-1-specific antisense inhibitor oligonucleotides (AMO-1, 80 μg in 100 μl) to ventricular myocytes, by multiple-site intramuscular injection of liposome-treated constructs into the myocardium. The injections were made before coronary artery occlusion to establish MI within the area equivalent to infracted zone (left ventricular front wall proximal to the apex). Shown are data expressed as % of incidence (mean±SE) and the actual incidence is indicated by the numbers above each individual bar. *p<0.05 vs. MI alone or HH Ctl; +p<0.05 vs. siRNAs; χ²-test. Panel (c) illustrates effects of the siRNAs on expression of GJA1 and KCNJ2 at the protein level (Cx43 and Kir2.1, respectively) determined by Western blot with membrane samples extracted from healthy rat hearts subjected to in vivo transfection of varying constructs. Left sub-panel shows examples of Western blot bands and right sub-panel shows mean±SE (n=5 independent samples for each group). The effects of the siRNAs on arrhythmias and Cx43 and Kir2.1 protein levels were qualitatively the same as those of miR-1. *p<0.05 vs. Ctl; unpaired Student t-test. Labels for the bars are the same below. Panel (d) illustrates the effects of the siRNAs on expression of GJA1 and KCNJ2 at the mRNA levels determined by real-time RT-PCR with the total RNA samples extracted from healthy rat hearts subjected to in vivo transfection of varying constructs. In opposition to miR-1, the mixed siRNAs significantly downregulated GJA1 and KCNJ2 mRNA levels. Data are mean±SE (n=5 independent samples for each group); *p<0.05 vs. Ctl; unpaired Student t-test. Panel (e) illustrates the selective knockdown of miR-1 by co-injected AMO-1. Data are mean±SE (n=5 independent samples for each group); *p<0.05; unpaired Student t-test. The above data demonstrate that direct knockdown of Cx43 and Kir2.1 by the siRNAs even when miR-1 levels had been reduced by AMO-1 produced similar effects on ischemic arrhythmogenesis as overexpression of miR-1, indicating that depression of Cx43 and Kir2.1 is a key link for the proarrhythmic action of miR-1.

FIG. 9 illustrates the specificity of the anti-miR-1 antisense inhibitor oligonucleotides (AMO-1) to target miR-1 and miR-1 actions on arrhythmias and the protein levels of Cx43 and Kir2.1, using a negative control AMO-1 (AMO-1 with ten mismatched nucleotides, Mis-AMO-1). Panels (a) and (b) illustrate anti-proarrhythmic effects of AMO-1 in ischemic rat hearts (MI, n=14) and control healthy rat hearts (HH, n=14). In vivo gene transfer was performed to deliver miR-1 (50 μg in 100 μl), together with AMO-1 or Mis-AMO-1 (80 μg in 100 μl) to ventricular myocytes, by multiple-site intramuscular injection of liposome-treated constructs into the myocardium. The injections were made before coronary artery occlusion to establish MI within the area equivalent to infracted zone (left ventricular front wall proximal to the apex). Shown are data expressed as % of incidence (mean±SE) and the actual incidence is indicated by the numbers above each individual bar. While AMO-1 efficiently reversed the proarrhythmic effects of miR-1, Mis-AMO-1 failed to do so. *p<0.05 vs. MI alone or HH Ctl; +p<0.05 vs. miR-1; c2-test. Panel (c) illustrates epicardial conduction velocity (mean±SE) measured in isolated Langendorff-perfused hearts with MI (n=7) or HH (n=6) rats. The constructs were delivered by direct intramuscular injection as described above, followed by coronary artery occlusion. The heart was isolated 12 h after MI and mounted to the Langendorff perfusion apparatus for measuring conduction velocity. *p<0.05 vs. MI alone or Ctl; +p<0.05 vs. miR-1 alone; unpaired Student t-test. (d) Resting membrane potential (mean±SE) measured by standard microelectrode techniques in tissue strips isolated from ischemic zone and boarder zone (BZ) of MI hearts (n=5) and HH (n=6) following injection delivery of varying constructs as described above. *p<0.05 vs. MI alone or Ctl; +p<0.05 vs. miR-1 alone; unpaired Student t-test. Panel (e) illustrates the selective effects of AMO-1 on miR-1-induced repression of Cx43 and Kir2.1 expression at the protein level, determined by Western blot with membrane samples extracted from healthy rat hearts subjected to in vivo transfection of varying constructs. Data are mean±SE (n=5 independent samples for each group). *p<0.05 vs. Ctl, +p<0.05 vs. miR-1 alone; unpaired Student t-test. Panel (f) illustrates the selective knockdown of miR-1 by co-transfected AMO-1. Data are mean±SE (n=5 independent samples for each group); *p<0.05 vs. Ctl, +p<0.05 vs. miR-1 alone; unpaired Student t-test. The data illustrated in this Figure indicate the specificity of the AMO-1 used in our study.

FIG. 10 illustrates the lack of effect of miR-1 on KCNH2 (encoding HERG K+ channel) expression, serving as negative control experiments for GJA1 and KCNJ2. Panel (a) illustrates Western blot analysis of HERG protein levels using the polyclonal anti-HERG antibody (Santa Cruz)(13). Protein samples were isolated from various regions of rat hearts of experimental myocardial infarction (12 h): NIZ, non-ischemic zone; IZ, ischemic zone; AP, antigenic peptide (pretreatment to neutralize the antibody). Shown are mean±SE from 6 Ctl and 7 CAD; *p<0.05 vs. Ctl; unpaired Student t-test. Panel (b) illustrates luciferase activity measured under various conditions in HEK293 cells. Cells were transfected with wild-type luciferase reporter plasmid or chimeric vectors containing luciferase gene followed by the 3′UTR of KCNH2 gene (GenBank Accession No. HSU04270), along with various concentrations of miR-1 or 100 nM AMO-1.

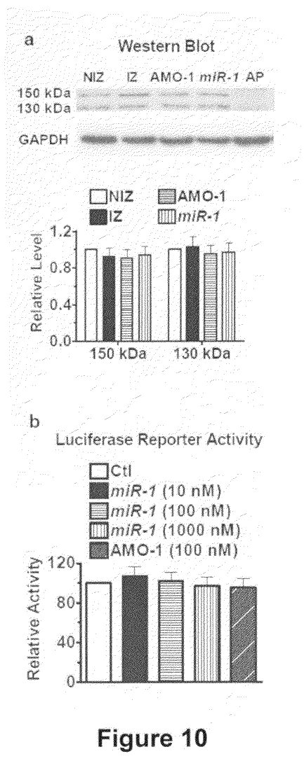

FIG. 11 illustrates the verification of effectiveness of miRNAs and AMO-1 used in the present study. Panel (a) illustrates the verification of interactions between rat miR-1 (100 nM) and the 3′UTRs of rat GJA1 and KCNJ2 genes in HEK293 cells, determined by luciferase reporter activity. Cells (1×10⁵/well) were transfected with 1 mg of varying constructs with lipofectamine 2000 (Invitrogen) following the manufacturer's instruction and the transfection took place 24 h after starvation of cells in serum-free medium. MT miR-1 (100 nM) failed to affect luciferase activity with WT 3′UTR of GJA1 or KCNJ2, but it elicited similar repressing effects on luciferase reporter gene as WT miR-1 did when the 3′UTRs were also mutated to match the MT miR-1 sequence. AMO-1 concentration used was 100 nM. Panel (b) illustrates Luciferase reporter activities showing the interactions of miR-1 and miR-133 with their respective exact binding sequences (standards). Data are presented as means ±SEM (n=5, 5, 5, and 4 batches of cells, respectively). *p<0.05 vs. Ctl; +p<0.05 vs. WT miR-1 or WT miR-133. miR-1 and miR-133 standards were used in which the complementary sequences of miR-1 and miR-133 were cloned downstream of luciferase gene in the pMIR-REPORT™ luciferase miRNA expression reporter vector (Ambion, Inc.). With these constructs, the uptake and activities of transfected miRNAs was confirmed. These experiments demonstrated that co-transfection of miR-1 and miR-1 standards or miR-133 and miR-133 standards into HEK293 cells nearly abolished the luciferase activities seen with transfection of miR-1 or miR-133 standards alone. The luciferase expression was unaffected if miR-1 had been co-transfected with miR-133 standards or if miR-133 had been co-transfected with miR-1 standards.

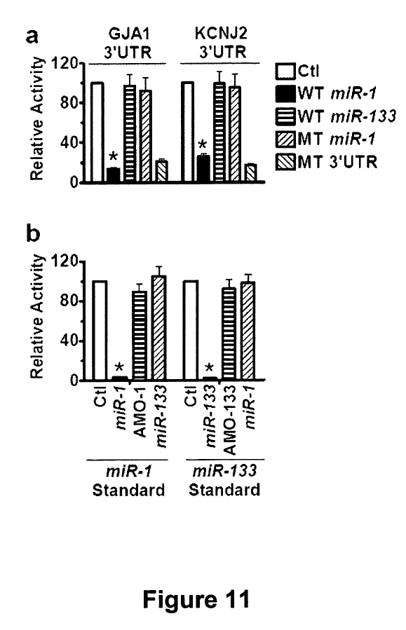

FIG. 12 is a Table showing the sequences disclosed herein, along with accession numbers, species information, and SEQ ID NOs.

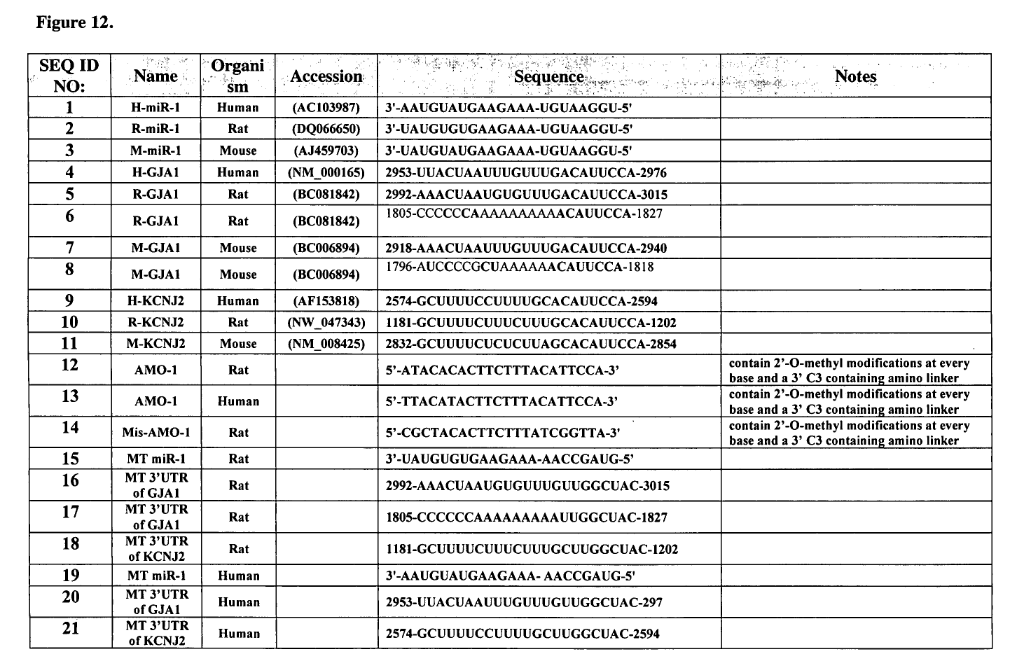

### DETAILED DESCRIPTION

The following definitions are provided for clarity and illustrative purposes only, and are not intended to limit the scope of the invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

“Mammal” refers to all known mammals and includes both human and veterinary subjects.

As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human.

In accordance with the present invention, there may be numerous tools and techniques that are well within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J., and Animal Cell Culture (Freshney, ed.:1986).

Common abbreviations correspond to units of measure, techniques, properties or compounds as follows: “min” means minutes, “h” means hour(s), “μL” means microliter(s), “mL” means milliliter(s), “mM” means millimolar, “M” means molar, “mmole” means millimole(s), “kb” means kilobase, and “bp” means base pair(s). “Polymerase chain reaction” is abbreviated PCR; “Reverse transcriptase polymerase chain reaction” is abbreviated RT-PCR; and “Sodium dodecyl sulfate” is abbreviated SDS.

microRNAs (miRNA) refer to single-stranded RNA molecules of about 21-23 nucleotides in length, which regulate gene expression. miRNAs are encoded by genes that are transcribed from DNA but not are not translated into protein); instead they are processed from primary transcripts known as pri-miRNA to short stem-loop structures called pre-miRNA and finally to functional miRNA. Mature miRNA molecules are partially complementary to one or more messenger RNA (mRNA) molecules, and one of their known functions is to downregulate gene expression.

The terms “protein” and “polypeptide” refer to compounds comprising amino acids joined via peptide bonds and are used interchangeably.

As used herein, where “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit the amino acid sequence to the deduced amino acid sequence, but include post-translational modifications of the deduced amino acid sequences, such as amino acid deletions, additions, and modifications such as glycolsylations and addition of lipid moieties.

The term “portion” when used in reference to a protein (as in “a portion of a given protein”) refers to fragments of that protein. The fragments may range in size from four amino acid residues to the entire amino sequence minus one amino acid.

The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences includes those obtained from the same or from different species of organisms.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “gene” refers to a nucleic acid (e.g., DNA or RNA) sequence that comprises coding sequences necessary for the production of an RNA, and/or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in 0 reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” may also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise a gene sequence that comprise cDNA forms of the gene; the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below.

The terms “region” or “portion” when used in reference to a nucleic acid molecule refer to a set of linked nucleotides that is less than the entire length of the molecule.

The term “strand” when used in reference to a nucleic acid molecule refers to a set of linked nucleotides which comprises either the entire length or less than or the entire length of the molecule.

The term “links” when used in reference to a nucleic acid molecule refers to a nucleotide region which joins two other regions or portions of the nucleic acid molecule; such connecting means are typically though not necessarily a region of a nucleotide. In a hairpin siRNA molecule, such a linking region may join two other regions of the RNA molecule which are complementary to each other and which therefore can form a double stranded or duplex stretch of the molecule in the regions of complementarity; such links are usually though not necessarily a single stranded nucleotide region contiguous with both strands of the duplex stretch, and are referred to as “loops.”

The term “linker” when used in reference to a multiplex siRNA molecule refers to a connecting means that joins two siRNA molecules. Such connecting means are typically though not necessarily a region of a nucleotide contiguous with a strand of each siRNA molecule; the region of contiguous nucleotide is referred to as a “joining sequence.”

The term “a polynucleotide having a nucleotide sequence encoding a gene” or “a polynucleotide having a nucleotide sequence encoding a gene” or “a nucleic acid sequence encoding” a specified RNA molecule or polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. This is also of importance in efficacy of RNA inhibition of gene expression or of RNA function.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence that is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. (U.S.A.) 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

When used in reference to a double-stranded nucleic acid sequence such as a cDNA or genomic clone, the term “substantially homologous” refers to any probe that can hybridize to either or both strands of the double-stranded nucleic acid sequence under conditions of low to high stringency as described above.

When used in reference to a single-stranded nucleic acid sequence, the term “substantially homologous” refers to any probe that can hybridize (i.e., it is the complement of) the single-stranded nucleic acid sequence under conditions of low to high stringency as described above.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “Tm” refers to the “melting temperature” of a nucleic acid. The melting temperature is the temperature at which a population of double-stranded nucleic acid molecules becomes half dissociated into single strands. The equation for calculating the Tm of nucleic acids is well known in the art. As indicated by standard references, a simple estimate of the T.sub.m value may be calculated by the equation: Tm=81.5+0.41 (% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tm.

As used herein the term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Low stringency conditions” when used in reference to nucleic acid hybridization comprise conditions equivalent to binding or hybridization at 42 C. in a solution consisting of 5×SSPE (43.8 g/l NaCl, 6.9 g/l NaH2PO.sub.4.H2O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)) and 100 ug/ml denatured salmon sperm DNA followed by washing in a solution comprising 5×SSPE, 0.1% SDS at 42 C. when a probe of about 500 nucleotides in length is employed.

“Medium stringency conditions” when used in reference to nucleic acid hybridization comprise conditions equivalent to binding or hybridization at 42 C. in a solution consisting of 5×SSPE (43.8 g/l NaCl, 6.9 g/l NaH2PO4.H2O and 1.85 g/l EDTA, pH adjusted to 7.4 with NaOH), 0.5% SDS, 5×Denhardt's reagent and 100 ug/ml denatured salmon sperm DNA followed by washing in a solution comprising 1.0×SSPE, 1.0% SDS at 42 C. when a probe of about 500 nucleotides in length is employed.

“High stringency conditions” when used in reference to nucleic acid hybridization comprise conditions equivalent to binding or hybridization at 42 C. in a solution consisting of 5×SSPE (43.8 g/l NaCl, 6.9 g/l NaH2PO4.H2O and 1.85 g/l EDTA, pH adjusted to 7.4 with NaOH), 0.5% SDS, 5×Denhardt's reagent and 100 .mu.g/ml denatured salmon sperm DNA followed by washing in a solution comprising 0.1×SSPE, 1.0% SDS at 42 C. when a probe of about 500 nucleotides in length is employed.

It is well known that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q_replicase, MDV-1 RNA is the specific template for the replicase (Kacian et al., Proc. Natl. Acad. Sci. USA, 69:3038 (1972)). Other nucleic acids will not be replicated by this amplification enzyme. Similarly, in the case of T7 RNA polymerase, this amplification enzyme has a stringent specificity for its own promoters (Chamberlin et al., Nature, 228:227 (1970)). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 (1989)). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

The term “amplifiable nucleic acid” refers to nucleic acids that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of .sup.32P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “reverse-transcriptase” or “RT-PCR” refers to a type of PCR where the starting material is mRNA. The starting mRNA is enzymatically converted to complementary DNA or “cDNA” using a reverse transcriptase enzyme. The cDNA is then used as a “template” for a “PCR” reaction

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and, where the RNA encodes a protein, into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “RNA function” refers to the role of an RNA molecule in a cell. For example, the function of mRNA is translation into a protein. Other RNAs are not translated into a protein, and have other functions; such RNAs include but are not limited to transfer RNA (tRNA), ribosomal RNA (rRNA), and small nuclear RNAs (snRNAs). An RNA molecule may have more than one role in a cell.

The term “inhibition” when used in reference to gene expression or RNA function refers to a decrease in the level of gene expression or RNA function as the result of some interference with or interaction with gene expression or RNA function as compared to the level of expression or function in the absence of the interference or interaction. The inhibition may be complete, in which there is no detectable expression or function, or it may be partial. Partial inhibition can range from near complete inhibition to near absence of inhibition; typically, inhibition is at least about 50% inhibition, or at least about 80% inhibition, or at least about 90% inhibition.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Exemplary constitutive plant promoters include, but are not limited to SD Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098), and ubi3 (see e.g., Garbarino and Belknap, Plant Mol. Biol. 24:119-127 (1994)) promoters. Such promoters have been used successfully to direct the expression of heterologous nucleic acid sequences in transformed plant tissue.

In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York (1989) pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI/BclI restriction fragment and directs both termination and polyadenylation (Sambrook, supra, at 16.6-16.7).

The term “vector” refers to nucleic acid molecules that transfer DNA segment(s) from one cell to another. The term “vehicle” is sometimes used interchangeably with “vector.” A vector may be used to transfer an expression cassette into a cell; in addition or alternatively, a vector may comprise additional genes, including but not limited to genes which encode marker proteins, by which cell transfection can be determined, selection proteins, be means of which transfected cells may be selected from non-transfected cells, or reporter proteins, by means of which an effect on expression or activity or function of the reporter protein can be monitored.

The term “expression cassette” refers to a chemically synthesized or recombinant DNA molecule containing a desired coding sequence and appropriate nucleic acid sequences necessary for the expression of the operably linked coding sequence either in vitro or in vivo. Expression in vitro includes expression in transcription systems and in transcription/translation systems. Expression in vivo includes expression in a particular host cell and/or organism. Nucleic acid sequences necessary for expression in prokaryotic cell or in vitro expression system usually include a promoter, an operator (optional), and a ribosome binding site, often along with other sequences. Eukaryotic in vitro transcription systems and cells are known to utilize promoters, enhancers, and termination and polyadenylation signals. Nucleic acid sequences necessary for expression via bacterial RNA polymerases, referred to as a transcription template in the art, include a template DNA strand which has a polymerase promoter region followed by the complement of the RNA sequence desired. In order to create a transcription template, a complementary strand is annealed to the promoter portion of the template strand.

The term “expression vector” refers to a vector comprising one or more expression cassettes. Such expression cassettes include those of the present invention, where expression results in an siRNA transcript.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, bacterial infection, viral infection, biolistics (i.e., particle bombardment) and the like. The terms “transfect” and “transform” (and grammatical equivalents, such as “transfected” and “transformed”) are used interchangeably.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 (1973)), has been modified by several groups to optimize conditions for

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “transgene” as used herein refers to a foreign gene that is placed into an organism by introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, .beta.-galactosidase, alkaline phosphatase, and horse radish peroxidase.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “antisense” when used in reference to DNA refers to a sequence that is complementary to a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The terms “nucleotide” and “base” are used interchangeably when used in reference to a nucleic acid sequence.

The term “strand selectivity” refers to the presence of at least one mismatch in either an antisense or a sense strand of a RNA molecule. The presence of at least one mismatch in an antisense strand results in decreased inhibition of target gene expression.

The term “cellular destination signal” is a portion of an RNA molecule that directs the transport of an RNA molecule out of the nucleus, or that directs the retention of an RNA molecule in the nucleus; such signals may also direct an RNA molecule to a particular subcellular location. Such a signal may be an encoded signal, or it might be added post-transciptionally.

The term “enhancing the function” when used in reference to an RNA molecule means that the effectiveness of an RNA molecule in silencing gene expression is increased. Such enhancements include but are not limited to increased rates of formation of an RNA molecule, decreased susceptibility to degradation, and increased transport throughout the cell. An increased rate of formation might result from a transcript which possesses sequences that enhance folding or the formation of a duplex strand.

The term “RNA interference” or “RNAi” refers to the silencing or decreasing of gene expression by one or more RNAs. It is the process of sequence-specific, post-transcriptional gene silencing in animals and plants, initiated by RNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “posttranscriptional gene silencing” or “PTGS” refers to silencing of gene expression in plants after transcription, and appears to involve the specific degradation of mRNAs synthesized from gene repeats.

The term “overexpression” refers to the production of a gene product in transgenic organisms that exceeds levels of production in normal or non-transformed organisms. The term “cosuppression” refers to the expression of a foreign gene that has substantial homology to an endogenous gene resulting in the suppression of expression of both the foreign and the endogenous gene. As used herein, the term “altered levels” refers to the production of gene product(s) in transgenic organisms in amounts or proportions that differ from that of normal or non-transformed organisms.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots).

The terms “Southern blot analysis” and “Southern blot” and “Southern” refer to the analysis of DNA on agarose or acrylamide gels in which DNA is separated or fragmented according to size followed by transfer of the DNA from the gel to a solid support, such as nitrocellulose or a nylon membrane. The immobilized DNA is then exposed to a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al. (1989) Molecular Cloning: A Laboratory Manual, Cold Spring Harbor Press, NY, pp 9.31-9.58).

The term “Northern blot analysis” and “Northern blot” and “Northern” as used herein refer to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al. (1989) supra, pp 7.39-7.52).

The terms “Western blot analysis” and “Western blot” and “Western” refers to the analysis of protein(s) (or polypeptides) immobilized onto a support such as nitrocellulose or a membrane. A mixture comprising at least one protein is first separated on an acrylamide gel, and the separated proteins are then transferred from the gel to a solid support, such as nitrocellulose or a nylon membrane. The immobilized proteins are exposed to at least one antibody with reactivity against at least one antigen of interest. The bound antibodies may be detected by various methods, including the use of radiolabeled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNA s which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

The methods and compositions of the present invention can be used alone or in conjunction with other tests known in the art for the “diagnosis” and/or detection of a cardiac disorder. The methods of present invention can be used alone or in conjunction with routine tests as an aid in diagnosis of cardiac pathologies.

The diagnostic methods of the present invention can be used alone or in conjunction with well-known tests to diagnose a disease or disorder within the context of the present invention. As but a further example, blood tests, ECG, and/or angioplasty are routinely used to diagnose or confirm a diagnosis and as well to determine the amount, extent and severity of damage to the heart. The diagnostic methods of the present invention can be used alone or in conjunction with these common tests in determining the amount, extent, or severity of damage to the heart.

“Treating” or “treatment” of a state, disorder or condition includes:

(1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or

(2) inhibiting the state, disorder or condition, i.e., arresting, reducing or delaying the development of the disease or a relapse thereof (in case of maintenance treatment) or at least one clinical or sub-clinical symptom thereof; or

(3) relieving the disease, i.e., causing regression of the state, disorder or condition or at least one of its clinical or sub-clinical symptoms.

The benefit to a subject to be treated is either statistically significant or at least perceptible to the patient or to the physician.

A “therapeutically effective amount” means the amount of a compound or composition that, when administered to a mammal for preventing or treating a state, disorder or condition, is sufficient to effect such prevention or treatment. The “therapeutically effective amount” of the compositions disclosed herein will vary depending on the compound or composition, the disease and its severity and the age, weight, physical condition and responsiveness of the animal to be treated. It is well within the ability of those skilled in the art to determine proper dosages to result in a therapeutically effective amount based on the type of mammal (species), as well as a mammal's physical characteristics (weight, fat content, etc.) and the desired result.

A “prophylactically effective amount” refers to an amount effective, at dosages and for periods of time necessary, to achieve the desired prophylactic result, such as preventing a disease or condition, including cardiac diseases or conditions. Typically, but not necessarily, since a prophylactic dose is used in subjects prior to or at an earlier stage of disease, the prophylactically effective amount will be less than the therapeutically effective amount.

The dosage of the therapeutic formulation will vary widely, depending upon the nature of the cardiac disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. Dose for various mammals can be extrapolated from data obtained in the experiments provided herein.

The compositions of the invention can be formulated for administration in any convenient way for use in mammalian, human, or veterinary medicine.

The pharmaceutical compositions may be in conventional forms, for example, capsules, tablets, aerosols, solutions, suspensions or products for topical application.

The route of administration may be any route which effectively transports the active compound of the invention to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment).

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir can be used in cases where a sweetened vehicle can be employed.

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

Administering RNA to a subject may also occur by directly exposing the subject a naked oligonucleotide, sense molecule, antisense molecule, or a suitable vector, or providing these materials to a subject in a conventional manner (e.g., oral or parenteral). Techniques to overexpress RNAs at the cellular level can also be used to administer RNA. Further, transient expression systems that use viral or liposomal delivery can be employed for administering large quantities of RNAs. Methods of synthesizing and delivering RNA to cells for observed effect are known in the art, for example, those synthesis and delivery methods disclosed in U.S. Patent Publication 2006/0063174 (hereby incorporated by reference in its entirety).

Recombinant methods for producing nucleic acids, including RNAs in a cell, are well known to those of skill in the art. These include the use of vectors (viral and non-viral), plasmids, cosmids, and other vehicles for delivering a nucleic acid to a cell, which may be the target cell or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice.

Suitable pharmaceutically acceptable excipients include, but are not limited to, diluents, binding agents, lubricants, glidants, disintegrants, and coloring agents. Other components such as preservatives, stabilizers, dyes and flavoring agents may be included in the dosage form. Examples of preservatives include sodium benzoate, ascorbic acid and esters of p-hydroxybenzoic acid. Antioxidants and suspending agents may be also included.

Pharmaceutically acceptable excipients, diluents, and carriers for therapeutic use are known in the pharmaceutical art, and are described, for example, in Remington: The Science and Practice of Pharmacy, Lippincott Williams & Wilkins (A.R. Gennaro edit. 2005).

MiR-1 has been implicated in determination of the differentiated state and in myogenesis (5,6). Increasing expression of miR-1 was found in neonatal hearts, and substantially higher levels are maintained in adult hearts (4-6), indicating that it may have other cellular and pathophysiological functions in addition to myogenesis. One of the questions was asked is whether miR-1 is involved in pathological processes relevant to human cardiac disease, besides its role in regulating development.

The experiments described below suggest methods for preventing or reversing cardiovascular conditions by interfering with miR-1 and are indicative of results that may be obtained in vitro and in vivo in any suitable mammal.

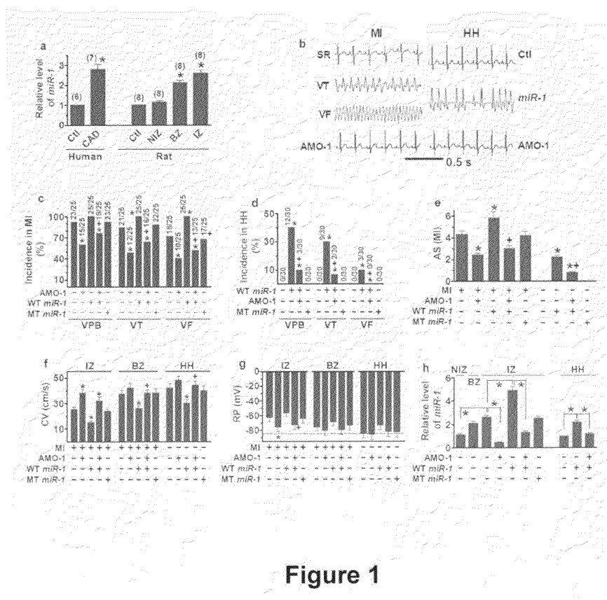

In a pilot study, it was found that miR-1 level was robustly elevated (by ˜2.8 folds) in RNA samples from individuals suffered from coronary artery disease (CAD, as seen in Table 1) compared with those of healthy human hearts (HH) (FIG. 1, panel a). To explore if this increase has any protective or detrimental consequence, we repeated the same measurements with RNA samples isolated from ischemic myocardium of the rat hearts subjected to experimental myocardial infarction (MI) for 12 h and our data showed a similar increase (˜2.6 folds) in miR-1 level in ischemic zone but not in non-ischemic zone (FIG. 1, panel a). This time point (12 h) corresponds to the peri-infarction period in CAD individuals during which phase II ischemic arrhythmias occur frequently, and understanding and treatment of phase II arrhythmias represent a major challenge (7,8). It has been well established that 2′-O-methyl-modified antisense oligoribonucleotides (AMO) act as specific and irreversible inhibitors of miRNA function and when administered intravenously knockdown target miRNAs in multiple organs including heart (9,10). We delivered an miR-1-specific AMO (AMO-1) into the infarcted myocardium by in vivo gene transfer (11-13) and strikingly, AMO-1 treatment significantly (p<0.05) suppressed arrhythmias as indicated by the reduced incidence of ventricular premature beat (PVB), ventricular tachycardia (VT) and ventricular fibrillation (VF) (FIG. 1, panels bace). In contrast, introduction of exogenous miR-1 into the infarcted myocardium promoted ischemic arrhythmias whilst mutant miR-1 failed to cause any arrhythmias. Moreover, co-injection of miR-1 with AMO-1 prevented arrhythmogenesis. These data strongly indicate that miR-1 is an arrhythmogenic/proarrhythmic factor detrimental to ischemic heart. We further demonstrated that delivery of exogenous miR-1 into healthy hearts (HH) was also arrhythmogenic; miR-1 “overexpression” induced VPB and VT which were otherwise absent without transfection of miR-1 (FIG. 1, panels b,d,e).

Arrhythmias are generally caused by abnormal automaticity, conduction, repolarization or any combination of these mechanisms. In diseased hearts, regional changes in electrophysiology can result in nonuniform anisotropy of impulse propagation and numerous investigations have highlighted the importance of anisotropic reentry in the formation of arrhythmias. We found that miR-1 “overexpression” significantly (p<0.05) widened QRS complex and prolonged QT interval (Table 2), indicative of cardiac conduction slowing, whereas AMO-1 narrowed it. This was indeed confirmed by reduced conduction velocity (CV), measured in isolated hearts, induced by miR-1 in MI and HH myocardium and the restoration of CV by AMO-1 (FIG. 1, panel 0. The resting membrane potential, determined in isolated tissue strips from left ventricular wall, was depolarized (FIG. 1, panel g), indicating that inward rectifier K+ current (IK1) was impaired.

Quantification of miR-1 in rat myocardium under various conditions verified the inverse relationships between miR-1 level and arrhythmogenesis, cardiac conduction disturbance and membrane potential abnormality and confirmed the silencing of both endogenous and exogenous miR-1 by AMO-1 (FIG. 1, panel h).

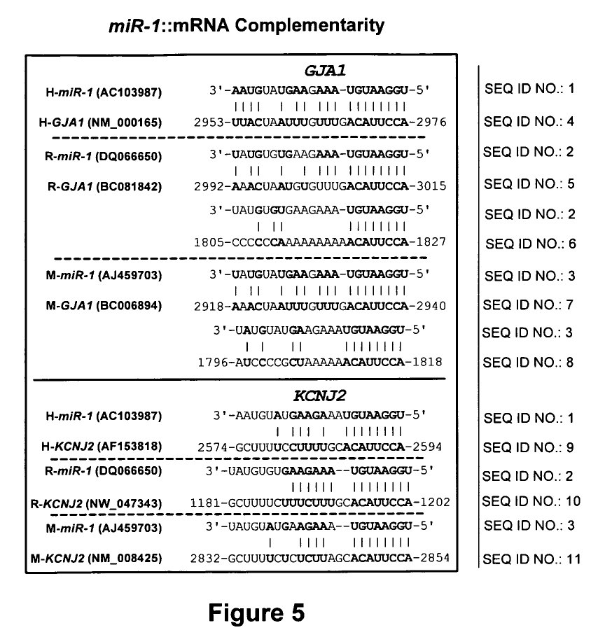

The above results suggest that miR-1 acts on ion channel genes to cause conduction slowing, membrane depolarization and arrhythmogenic/proarrhythmic effects. To test this notion, we first identified two relevant targets for miR-1: GJA1 and KCNJ2, among all known ion channel genes. GJA1 encoding connexin 43 (Cx43), the major cardiac gap junction channel underlying junctional current responsible for intercellular conductance in ventricle14 and KCNJ2 encoding Kir2.1, the major K+ channel subunit underlying IK1 responsible for setting and maintaining cardiac resting membrane potentially. The 3′UTR regions of GJA1 and KCNJ2 both contain stretches of eight nucleotides perfectly complementary to the first eight nucleotides from the 5′ end of miR-1 (FIG. 5).

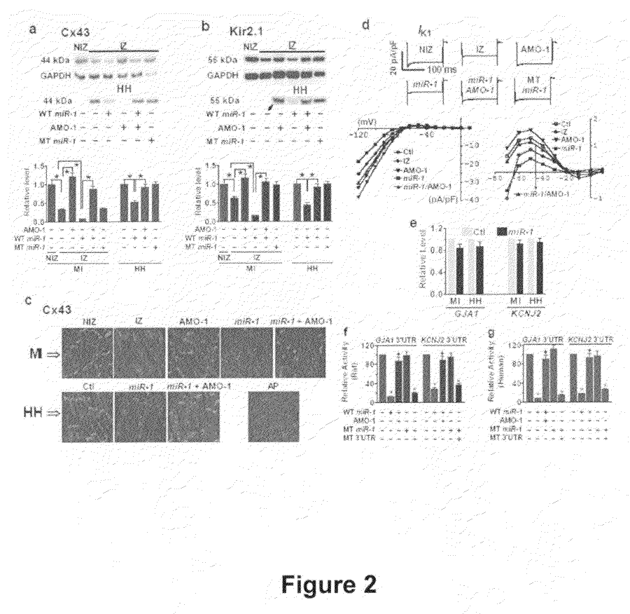

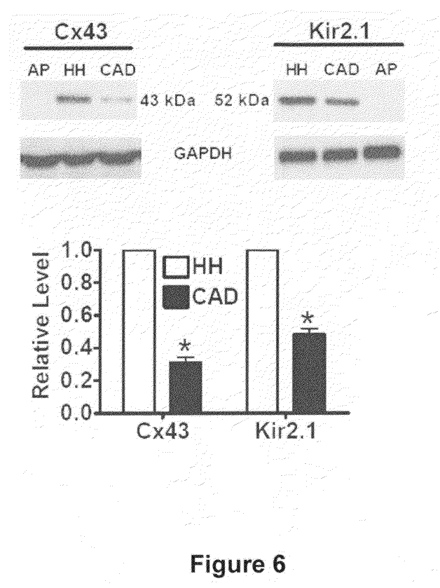

To verify that GJA1 and KCNJ2 are indeed the cognate targets of miR-1 for post-transcriptional repression, we determined the effects of miR-1 on the expression of these genes at protein levels by Western blot. Our experiments revealed that Cx43 and Kir2.1 levels were both significantly (p<0.05) diminished in MI rat, consistent with our previous findings (16), and the reduction was reversed by pretreatment with AMO-1. More importantly, miR-1 produced significantly (p<0.05) decreases of Cx43 to ˜8% of the control level (FIG. 2, panel a) and of Kir2.1 to ˜16% of the normalized expression (FIG. 2, panel b). Knockdown of Cx43 by miR-1 was also confirmed by immunohistochemistry (FIG. 2, panel c) and that of Kir2.1 was verified by whole-cell patch-clamp recording of IK1 (FIG. 2d). When injected with mutant miR-1 (MT miR-1), the repression of GJA1 and KCNJ2 were hardly seen, and co-application of miR-1 with AMO-1 nearly abolished the repressing effects of miR-1 on GJA1 and KCNJ2 (FIG. 2, panels a and b). By comparison with the data in FIG. 1, panel h, a clearly concordant inverse correlation between miR-1 level and Cx43 and Kir2.1 protein levels can be seen. Reduced protein levels of Cx43 and Kir2.1 were also consistently found in human hearts with CAD (FIG. 6).

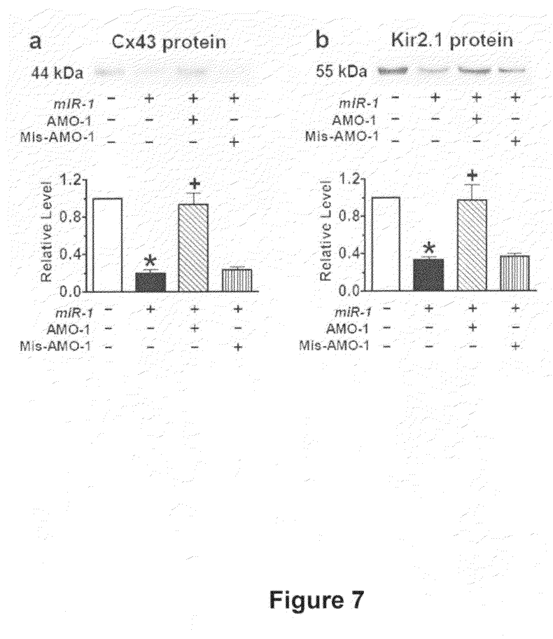

To verify the specificity and effects of AMO-1, all experiments involving AMO-1 were repeated with comparison to a negative control AMO-1 that carries ten mismatched nucleotides in the AMO-1 (as described in the Supplementary Methods section hereinbelow). The data clearly showed that the negative control AMO-1 failed to reduce miR-1 level and failed to prevent downregulation of Cx43 and Kir2.1 protein levels and ischemic arrhythmias caused by miR-1 application (FIG. 7).

Because it has been shown that miRNAs can also down-regulate a specific target by affecting mRNA stability (1), we subsequently investigated effects of miR-1 on mRNA levels of GJA1 and KCNJ2. Our data demonstrated no effect of miR-1 on mRNA stability of these genes (FIG. 2, panel e).

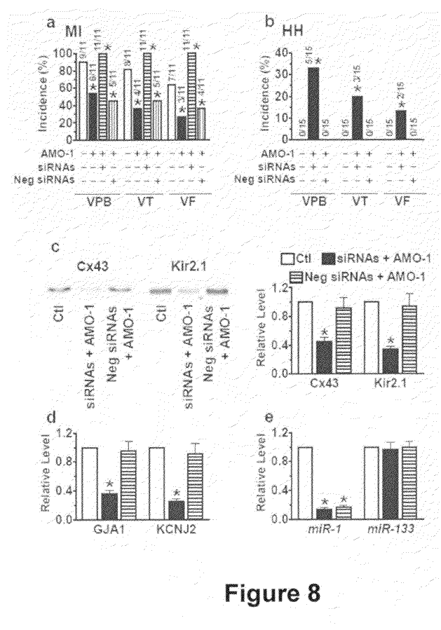

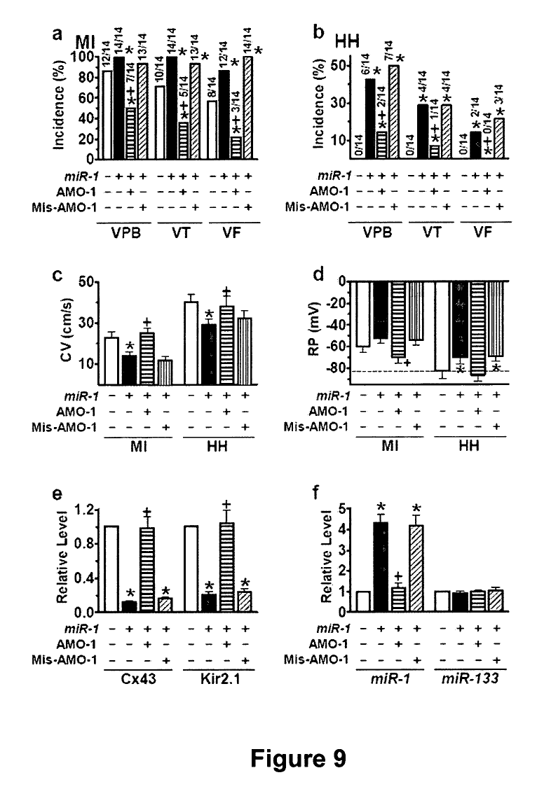

Previous studies suggest that miRNA-binding sites are transferable and sufficient for conferring miRNA-dependent gene silencing. We inserted the 3′UTRs of GJA1 and KCNJ2 into the 3′UTR of a luciferase reporter plasmid containing a constitutively active promoter in order to determine the effects of miR-1 on reporter expression. Co-transfection of the plasmid with miR-1 (FIG. 2, panels f,g) into HEK293 cells consistently demonstrated smaller luciferase activities relative to the plasmid alone, whereas the mutant miR-1 failed to elicit any effects. Transfection of AMO-1 eliminated the silencing effects of miR-1 on the activities of the wild-type GJA1- or KCNJ2-luciferase chimeric vector and target sequences. On the other hand, mutant miR-1 remarkably repressed translation of luciferase transcripts containing the complementary mutant GJA1 or KCNJ2 3′-UTR. The same results were obtained with both rat (FIG. 2, panel f) and human (FIG. 2, panel g) miR-1s and GJA1/KCNJ2 3′UTRs. Negative controls experiments with KCNH2 encoding HERG K+ channel were also performed (FIG. 8). The oligonucleotides (miR-1, miR-133, and AMO-1) were validated for their respective activities using the luciferase reporter system carrying the exact binding sequences for miR-1 and miR-133 (FIG. 8). Finally, the uptake and distribution of miR-1 and AMO-1 after in vivo transferring procedures were examined (FIG. 9).

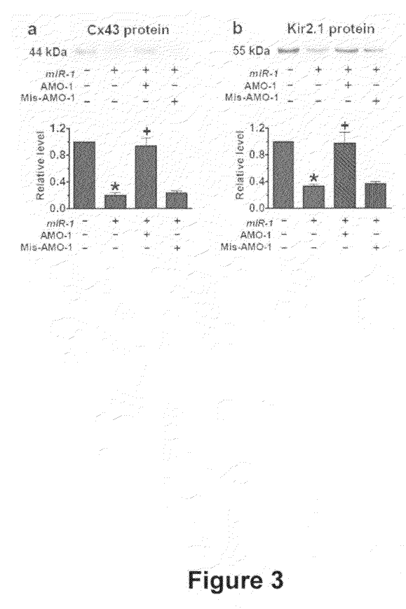

We also confirmed the ability of miR-1 to downregulate Cx43 and Kir2.1 protein levels in isolated neonatal rat ventricular myocytes in culture. Co-transfection of miR-1 with AMO-1 abolished the repression of Cx43 and Kir2.1 expression at the protein level and actually increased the protein levels above control values (presumably, but non-limitingly, because AMO-1 removed the basal repression produced by endogenous miR-1) (FIG. 3).

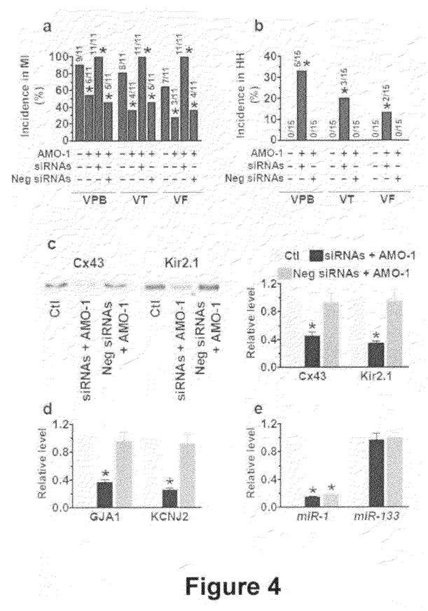

If the proarrhythmic action of miR-1 was indeed associated with repression of Cx43 and Kir2.1 proteins, then direct downregulation of Cx43 and Kir2.1 should also be able to induce arrhythmias. We therefore further investigated the link between miR-1 and Cx43 and Kir2.1 to ischemic arrhythmias using the RNAi techniques. Co-injection of the siRNA targeting Cx43 and Kir2.1, respectively, with AMO-1 into the myocardium of ischemic rat hearts induced significant arrhythmias despite that the miR-1 level was downregulated by co-applied AMO-1 (FIG. 4). The results are consistent with previous findings in Cx43-knockout17,18 or Kir2.1-knockout mice19.

The present study revealed the pathological role of miRNA in heart, i.e. proarrhythmic and arrhythmogenic effects of miR-1 and a novel aspect of cellular functions of miR-1: repressing ion channel genes. Our data also presents the first demonstration that (1) pathological elevation of miR-1 in CAD individuals and experimental MI rats, (2) gene silencing of GJA1 and KCNJ2 by miR-1 via repressing translation with little effects on mRNA cleavage, which is likely a mechanism underlying its proarrhythmic/arrhythmogenic potential, and (3) inhibition of endogenous miR-1 and ischemic arrhythmias by AMO-1, a potential novel approach for antiarrhythmic therapy. We also observed miR-1 overexpression in myocardium of ischemic reperfusion with miRNA microarray methods (data not shown). Intriguingly, expression of miR-1 may be differentially regulated under different pathological conditions. A recent study using miRNA microarray approach showed that miR-1 level was downregulated in aortic constriction-induced hypertrophy of a mouse model20. This downregulation may be necessary for induction of hypertrophy since it may relieve growth-related target genes from repressive influence by miR-1. However, an earlier study in which the same model and the same approach wee used failed to observe reduction of miR-121. Thus, the exact role of miR-1 in hypertrophy remained unclear.

Methods

Rat Model of Myocardial Infarction. Male Wistar rats of 230-270 g were randomly divided into control and myocardial infarction (MI) groups. Myocardial infarction was established as previously described (22). The rats were anesthetized with diethyl ether and placed in the supine position with the upper limbs taped to the table. A 1-1.5 cm incision was made along the left side of the sternum. The muscle layers of the chest wall were bluntly dissected to avoid bleeding. The thorax was cut open at the point of the most pronounced cardiac pulsation and the right side of the chest were pressed to push the heart out of the thoracic cavity. The left anterior descending (LAD) coronary artery was occluded and then the chest was closed back. All surgical procedures were performed under sterile conditions. Twelve hours after occlusion, the heart was removed for Langendorff perfusion experiments or the tissues within ischemic zone (IZ), boarder zone (BZ) and non-ischemic zone (NIZ) distal to the ischemic zone were dissected for measurement of miR-1, GJA1 and KCNJ2 levels. Control animals underwent open-chest procedures without coronary artery occlusion. Use of animals was in accordance with the regulations of the ethic committees of Harbin Medical University.

Measurements of Infarct Area. The hearts were removed from the animals 12 h after infarction and ventricular tissues were dissected and kept overnight at −4° C. Frozen ventricles were sliced into 2 mm thick sections, and then incubated in 1% triphenyltetrazolium chloride at 37° C. in 0.2 M Tris buffer (pH 7.4) for 30 min. While the normal myocardium was stained brick red, the infarcted areas remained unstained. Size of the infarcted area was estimated by the volume and weight as a percentage of the left ventricle (22).

In vivo Gene Transfer. With the open chest described above, 50-100 μg in 100 ml of synthesized miR-1, mutant miR-1 (MT miR-1), AMO-1, Mis-AMO-1, siRNAs (targeting GJA1 and KCNJ2, respectively) or negative control siRNAs (Neg siRNAs), pretreated with lipofectamine 2000 (Invitrogen) was injected through a 26-gauge needle into the myocardium (11-13). The intramuscular injections were made in multiple-sites (˜10 sites) before coronary artery occlusion to establish MI within the area equivalent to infracted zone (left ventricular front wall proximal to the apex, ˜0.8 cm² area). Following injection, the heart was placed back into the thoracic cavity, the chest was closed with sutures, and the rat was allowed to recover. Experimental measurements were made 12 h after intramuscular injection and MI.

Conduction Studies. Conduction velocity (CV) was measured on the ventricular epicardial surface in perfused hearts, using the method described by Guerrero et al (18). Twelve hours after coronary artery occlusion and intramuscular injection, rat hearts were rapidly excised and placed in oxygenated cardioplegic solution at 4° C. Hearts were perfused with the same buffer via an aortic cannula at a flow rate of 1.0-1.2 ml/min while simultaneously being superfused with buffer at a flow rate of 12 ml/min. A temperature of 31° C. was chosen to slow the spontaneous heart rate, and thereby to facilitate pacing. This temperature is also expected to slow CV, and thus facilitate comparisons of CV under different conditions. A linear electrode array consisting of 16 bipolar pairs (interelectrode distance 200 mm) was placed on the anterior surface of each heart along the maximal apical-basal dimension. Care was taken to place the electrode array in the same location in each heart in an orientation roughly parallel to the left anterior descending coronary artery. After a 5-min stabilization interval, spontaneous and paced electrical activity was recorded. Paced beats (twice threshold at a basic cycle length of 300 ms) were initiated with a bipolar electrode at the ventricular apex. Electrograms were recorded on a multichannel computerized data acquisition system. Activation times were defined by determining the maximum absolute amplitude of each electrogram (peak criterion), and the average CV was calculated by linear regression relating interelectrode distance to activation times. The slope of the regression line was the average CV.

Synthesis of miRNAs and Sequences of miRNA Inhibitor.

Human and rat miR-1s (FIG. 5) and their respective mutant constructs (see below) were synthesized by Integrated DNA Technologies (IDT, Inc.). The sequence of AMO-1 is the exact antisense copies of their mature miRNA sequences (for rat: 5′-ATACACACTTCTTTACATTCCA-3′) (SEQ ID NO:12); for human: 5′-TTACATACTTCTTTACATTCCA-3′) (SEQ ID NO:13)), and the sequences of the mismatched AMO-1 (Mis-AMO-1 for negative control) carry ten mismatched nucleotides to miR-1 mainly at the 3′-end (for rat: 5′-CGCTACACTTCTTTATCGGTTA-3′) (SEQ ID NO:14). The AMO-1 and Mis-AMO-1 contain 2′-O-methyl modifications at every base and a 3′C3 containing amino linker.

Mutagenesis. Nucleotide-substitution mutations were carried out using direct oligomers synthesis for miR-1 (MT miR-1) and PCR-based methods for the 3′UTRs of GJA1 and KCNJ2 genes.

For Rat Genes:

MT miR-1:

(SEQ ID NO:15)

3′-UAUGUGUGAAGAAA-AACCGAUG-5′;
MT 3′UTR of GJA1:

(SEQ ID NO:16)

···2992-AAACUAAUGUGUUUGUUGGCUAC-3015···
and

(SEQ ID NO:17)

···1805-CCCCCCAAAAAAAAAUUGGCUAC-1827···;
3′UTR of KCNJ2:

(SEQ ID NO: 18)

···1181-GCUUUUCUUUCUUUGCUUGGCUAC-1202···.

For Human Genes:

MT miR-1:

(SEQ ID NO:19)

3′-AAUGUAUGAAGAAA-AACCGAUG-5′;
GJA1:

(SEQ ID NO:20)

···2953-UUACUAAUUUGUUUGUUGGCUAC-2976···;
KCNJ2:

(SEQ ID NO:21)

···2574-GCUUUUCCUUUUGCUUGGCUAC-2594···.

All constructs were sequencing verified.

Supplemental Methods

Rat Model of Myocardial Infarction

Male Wistar rats of 230-270 g were randomly divided into control and myocardial infarction (MI) groups. Myocardial infarction was established as previously described in reference (25). The rats were anesthetized with diethyl ether and were placed in the supine position with the upper limbs taped to the table. Chest skin was cleaned with 70% ethanol and a 1-1.5 cm incision was made along the left side of the sternum. The muscle layers of the chest wall were bluntly dissected to avoid bleeding. The thorax was cut open at the point of the most pronounced cardiac pulsation. Using forceps to widen the chest, the abdomen and the right side of the chest were pressed to push the heart out of the thoracic cavity. The left anterior descending (LAD) coronary artery was occluded and then the chest was closed back. All surgical procedures were performed under sterile conditions. Twelve hours after occlusion, the heart was removed for Langendorff perfusion experiments or the tissues within ischemic zone (IZ), boarder zone (BZ) and non-ischemic zone (NIZ) distal to the ischemic zone were dissected for measurement of miR-1, GJA1 and KCNJ2 levels. Control animals underwent open-chest procedures without coronary artery occlusion. Use of animals was in accordance with the regulations of the ethic committees of Harbin Medical University.

Measurements of Infarct Area

The hearts were removed from the animals 12 h after infarction and ventricular tissues were dissected and kept overnight at −4° C. Frozen ventricles were sliced into 2 mm thick sections, and then incubated in 1% triphenyltetrazolium chloride at 37° C. in 0.2 M Tris buffer (pH 7.4) for 30 min. While the normal myocardium was stained brick red, the infarcted areas remained unstained. Size of the infarcted area was estimated by the volume and weight as a percentage of the left ventricle, as described in reference (25).

Synthesis of miRNAs and Sequences of miRNA Inhibitor

Human and rat miR-1s, illustrated in FIG. 5, which are substantially identical except for the first nucleotide at the 3′ end, and their respective mutant constructs (see Mutagenesis section), were synthesized by Integrated DNA Technologies (IDT, Inc.). The sequences of anti-miR-1 antisense inhibitor oligonucleotides (AMO-1) are the exact antisense copies of their mature miRNA sequences (for rat: 5′-ATACACACTTCTTTACATTCCA-3′) (SEQ ID NO:12); for human: 5′-TTACATACTTCTTTACATTCCA-3′) (SEQ ID NO:13)), and the sequences of the mismatched AMO-1 (Mis-AMO-1 for negative control) carry ten mismatched nucleotides to miR-1 mainly at the 3′-end (for rat: 5′-CGCTACACTTCTTTATCGGTTA-3′) (SEQ ID NO:14). The AMO-1 and MIs-AMO-1, synthesized by IDT contain 2′-O-methyl modifications at every base and a 3′C3 containing amino linker.

Mutagenesis

Nucleotide-substitution mutations were carried out using direct oligomers synthesis for miR-1 (MT miR-1), and PCR-based methods for the 3′UTRs of GJA1 and KCNJ2 genes.

### For Rat Genes:

MT miR-1:

(SEQ ID NO: 15)

3′-UAUGUGUGAAGAAA-AACCGAUG-5′;
MT 3′UTR of GJA1:

(SEQ ID NO: 16)

···2992-AAACUAAUGUGUUUGUUGGCUAC-3015···
and

(SEQ ID NO: 17)

···1805-CCCCCCAAAAAAAAAUUGGCUAC-1827···;
MT 3′UTR of KCNJ2:

(SEQ ID NO: 18)

···1181-GCUUUUCUUUCUUUGCUUGGCUAC-1202···.

### For Human Genes,

MT miR-1:

(SEQ ID NO: 19)

3′-AAUGUAUGAAGAAA-AACCGAUG-5′;
GJA1:

(SEQ ID NO: 20)

···2953-UUACUAAUUUGUUUGUUGGCUAC-2976···;
KCNJ2:

(SEQ ID NO: 21)

···2574-GCUUUUCCUUUUGCUUGGCUAC-2594···.

All constructs were sequencing verified.

Small Interference RNA (siRNA)

The cassettes carrying the siRNAs targeting GJA1 and KCNJ2, respectively, were constructed using the sixpresse Mouse U6 PCR Vector System (Mirus Bio Corporation, Madison, Wis.) given by Prof. Guohao Chen (Cardiovascular Research Institute, Guangzhou, P. R. China) as a kind gift, according to the manufacturer's protocol. The Cx43-siRNA sequence used in our study is the same as that reported by Shao et al (26) and by Sanchez-Alvarez et al (27): 5′-GAAGTTCAAGTACGGGATT-3′ (SEQ ID NO: 19) targeting the intracellular loop of rat Cx43 from 398 to 416 (GenBank No. BC081842). The Kir2.1-siRNA sequence used in our study is the same as that reported by Rinne et al (28): 5′-GGTGTGTTACAGACGAGTG-3′ (SEQ ID NO: 20) corresponding to rat Kir2.1 from 443 to 461 (GenBank No. NM_{—}017296). These siRNAs were selected because their effectiveness has been verified by previous studies (26-28) and examined by cross-checking and reaching consensus with three companies that offer siRNA design: GenScript Corporation (Scotch Plains, N.J.), Qiagen (Mississauga, ON), and Ambion (Austin, Tex.).

In Vivo Gene Transfer

With the open chest described above, 50-100 μg in 100 μl of synthesized miR-1, mutant miR-1 (MT miR-1), AMO-1, Mis-AMO-1, siRNAs (targeting GJA1 and KCNJ2, respectively) or negative control siRNAs (Neg siRNAs), pretreated with lipofectamine 2000 (Invitrogen) was injected through a 26-gauge needle into the area equivalent to the infracted region proximal to apex of the heart, as described in references (29 and 30). Following injection, the heart was placed back into the thoracic cavity, the chest was closed with sutures, and the rat was allowed to recover. For experiments involving myocardial infarction, In vivo gene transfer was carried out right before coronary artery occlusion and experimental measurements were made 12 h after intramuscular injection and MI.

Quantification of mRNA and miRNA Levels

For quantification of GJA1 and KCNJ2 transcripts, conventional real-time RT-PCR was carried out with total RNA samples extracted from rat ventricular wall of experimental myocardial infarction and treated with DNase I. TaqMan quantitative assay of transcripts was performed with real-time two-step reverse transcription PCR (GeneAmp 5700, PE Biosystems), involving an initial reverse transcription with random primers and subsequent PCR amplification of the targets. Expression level of GAPDH was used as an internal control, as described in reference (31).

The mirVana™ qRT-PCR miRNA Detection Kit (Ambion) is a quantitative reverse transcription-PCR (qRT-PCR) kit enabling sensitive, rapid quantification of microRNA (miRNA) expression from total RNA samples, was used in conjunction with real-time PCR with SYBR Green I for quantification of miR-1 transcripts in our study, following the manufacturer's instructions. The total RNA samples were isolated with Ambion's mirvana miRNA Isolation Kit, from human left ventricular preparations from patients with myocardial infarction due to coronary artery disease (CAD) and from rat myocardium. Reactions contained mirVana qRT-PCR Primer Sets specific for human or rat miR-1s and human 5S rRNA as positive controls. qRT-PCR was performed on a GeneAmp 5700 thermocycler for 40 cycles. We first determined the appropriate cycle threshold (Ct) using the automatic baseline determination feature. We then performed dissociation analysis (melt-curve) on the reactions to identify the characteristic peak associated with primer-dimers in order to separate from the single prominent peak representing the successful PCR amplification of miR-1. Fold variations in expression of miR-1 between RNA samples were calculated. Human tissues were obtained from the Second Affiliated Hospital of Harbin Medical University under the procedures approved by the Ethnic Committee for Use of Human Samples of the Harbin Medical University and from the Reseau de tissus pour etudes biologiques (RETEB) tissue bank under the procedures approved by the Human Research Ethics Committee of the Montreal Heart Institute.

Cardiac Arrhythmias

Spontaneous arrhythmias were recorded with open chest rats with standard lead II ECG. The Curtis and Walker arrhythmia scoring method, described for example in reference (32) was employed. In brief, 0=no arrhythmia; 1=ventricular premature beats (VEB) and/or ventricular tachycardia (VT) of <10-s duration; 2=VES and/or VT of 11-30 s; 3=VES and/or VT of 31-90 s; 4=VES and/or VT of 91-180 s, or reversible ventricular fibrillation (VF) of <10 s; 5=VES and/or VT of >180 s, or reversible VF of >10 s; 6=irreversible VF. Incidence of arrhythmias of different sorts was calculated as percentage of animals with arrhythmias over the total number of animals used: 30 MI rats and 25 healthy rats (1).

Conduction Studies

Conduction velocity was measured on the ventricular epicardial surface in perfused hearts, using the method described by Guerrero et al in reference (33). Twelve hours after coronary artery occlusion and intramuscular injection, rat hearts were rapidly excised and placed in oxygenated cardioplegic solution at 4° C. Hearts were perfused with the same buffer via an aortic cannula at a flow rate of 1.0-1.2 ml/min while simultaneously being superfused with buffer at a flow rate of 12 ml/min. A temperature of 31° C. was chosen to slow the spontaneous heart rate, and thereby to facilitate pacing. This temperature was also expected to slow conduction, and thus facilitate comparisons of conduction velocity under different conditions. A linear electrode array consisting of 16 bipolar pairs (interelectrode distance 200 mm) was placed on the anterior surface of each heart along the maximal apical-basal dimension. Care was taken to place the electrode array in the same location in each heart in an orientation roughly parallel to the left anterior descending coronary artery. After a 5-min stabilization interval, spontaneous and paced electrical activity was recorded. Paced beats (twice threshold at a basic cycle length of 300 ms) were initiated with a bipolar electrode at the ventricular apex. Electrograms were recorded on a multichannel computerized data acquisition system. Activation times were defined by determining the maximum absolute amplitude of each electrogram (peak criterion), and the average conduction velocity was calculated by linear regression relating interelectrode distance to activation times. The slope of the regression line was the average conduction velocity.

Myocytes Isolation from Adult Rat Heart

The procedures were similar to the previously described method, as described for example in references (34 and 35). Adult rat hearts were removed and mounted on a modified Langendorff perfusion system for retrograde perfusion via the coronary circulation. The preparation was perfused with Ca²⁺-containing Tyrode's solution (in mM): NaCl 126, KCl 5.4, MgCl2 1, CaCl2 1.8, NaH₂PO₄ 0.33, glucose 10, and Hepes 10 (pH 7.4, with NaOH) at 37° C. until the effluent was clear of blood and then switched to Ca²⁺-free Tyrode's solution for 20 min at a constant rate of 12 ml/min, followed by perfusion with the same solution containing collagenase (type II, 10-150 kU/L) and 1% bovine serum albumin. The left ventricular epicardial layers within IZ, around IZ, and in NIZ areas were then excised from the softened hearts, minced, and placed in a KB medium (in mM): glutamic acid 70, taurine 15, KCl, 30, KH₂PO₄ 10, MgCl₂ 0.5, EGTA 0.5, glucose 10, and Hepes 10 (pH 7.4, with KOH) at 4° C. for 1 h before electrophysiological experiments.

Cell Isolation from Neonatal Rat Heart and Primary Cell Culture

Neonatal rat ventricular cardiomyocytes were isolated and cultured as described in reference (31). Briefly, 1-3 days old rats were decapitated and their hearts were aseptically removed. Their ventricles were dissected, minced and trypsinized overnight at 4° C. The next day, cells were dissociated with collagenase and pre-plated twice for 60 min at 37° C. The non-adherent cardiomyocytes were removed and plated in 24-well plates in DMEM/F-12 medium (Invitrogen) containing 10% FBS and 0.1 mM bromodeoxyuridine (Sigma). 1×10⁵ cells/well were seeded in 24-well plate for further experiments. This procedure yielded cultures with 90±95% myocytes, as assessed by microscopic observation of cell beating.

Whole-Cell Patch-Clamp Recording

Patch-clamp techniques have been described in detail elsewhere, for example in references (35 and 36). Currents were recorded in the whole-cell voltage-clamp mode, with an Axopatch-200B amplifier (Axon Instruments). Borosilicate glass electrodes had tip resistances of 1-3 MΩ when filled with the internal pipette solution. The pipette solution for K⁺ current recording contained (mM): 130 KCl, 1 MgCl₂, 5 Mg-ATP, 10 EGTA, and 10 HEPES (pH adjusted to 7.25 with KOH). The internal pipette solution for AP recording contained same components as for K⁺ currents recording, except that EGTA was 0.05 mM. 4-Aminopyridine (1 mM) was used to inhibit Iₜₒ and external glyburide (10 μM) plus internal Mg-ATP (5 mM) to prevent ATP-sensitive K⁺ current. I_{Na} and I_{Ca} were inactivated by holding the membrane at −20 mV. Experiments were conducted at 36±1° C. Junction potentials were zeroed before formation of the membrane-pipette seal and they were not corrected for our data analyses. Series resistance and capacitance were compensated and leak currents were subtracted.

I_{K1} was elicited by 200-ms pulses ranging from −120 mV to +10 mV with an increment of 10 mV from a holding potential of −20 mV (36). Since our study was designed for group comparisons of the experimental results, the currents were all recorded immediately after membrane rupture and series resistance compensation in order to minimize the possible time-dependent rundown, run-up, or negative shift of currents. Individual currents were normalized to the membrane capacity to control for differences in cell size, being expressed as current density pA/pF.

Western Blot

The protein samples were extracted from rat left ventricular wall or cultured neonatal rat ventricular myocytes, with the procedures essentially the same as described in detail in references (34 and 35). The protein content was determined with Bio-Rad Protein Assay Kit (Bio-Rad, Mississauga, ON, Canada) using bovine serum albumin as the standard. Protein sample (˜150 μg) was fractionated by SDS-PAGE (7.5%-10% polyacrylamide gels) and transferred to PVDF membrane (Millipore, Bedford, Mass.). The samples were incubated overnight at 4° C. with primary antibodies for connexin43 and Kir2.1 (Santa Cruz). Bound antibodies were detected using the chemiluminescent substrate (Western Blot Chemiluminescence Reagent Plus, NEN Life Science Products, Boston, USA). Western blot bands were quantified using Quantityone software by measuring the band intensity (Area×OD) for each group and normalizing to GAPDH (anti-GAPDH antibody from Research Diagnostics Inc) as an internal control. The final results are expressed as fold changes by normalizing the data to the control values.

Construction of Chimeric miRNA Binding Site-Luciferase Reporter Vectors

To generate reporter vectors bearing miRNA-binding sites, we generated match direct human and rat miR-1 sites, either wild-type or mutated (synthesized by Invitrogen), respectively, and the 3′UTRs of human and rat GJA1 and KCNJ2 genes including both either wild-type and mutated constructs (obtained by RT-PCR amplification) or the 3′UTR of KCNH2 (encoding HERG channel protein). These inserts were cloned into the multiple cloning sites in the pMIR-REPORT™ luciferase miRNA expression reporter vector (Ambion, Inc.). The sense and antisense strands of the oligonucleotides were annealed by adding 2 μg of each oligonucleotides to 46 μl of annealing solution (100 mM K-acetate, 30 mM HEPES-KOH, pH 7.4 and 2 mM Mg-acetate) and incubated at 90° C. for 5 min and then at 37° C. for 1 h. The annealed oligonucleotides were digested with HindIII and SpeI and used to ligate into HindIII and SpeI sites.

Cell Culture

The cell lines used in this study were all purchased from American Type Culture Collection (ATCC, Manassas, Va.). HEK293 cells (human embryonic kidney cell line) were cultured in Dulbecco's Modified Eagle Medium (DMEM), reference (37). The culture was supplemented with 10% fetal bovine serum and 100 μg/ml penicillin/streptomycin. HEK293 cells were used as these cells express minimal miR-1.

In Vitro Transfection and Luciferase Assay

After 24 h starvation in serum-free medium, cells (1×10⁵/well) were transfected with 1 μg miR-1, MT miR-1, AMO-1, Mismatched AMO-1 (Mis-AMO-1), siRNAs (targeting GJA1 and KCNJ2, respectively) or negative control siRNAs (Neg siRNAs) with lipofectamine 2000 (Invitrogen), according to the manufacturer's instructions. Experiments were performed 12 h after transfection.

For luciferase assay, cells were similarly transfected with 1 μg PGL3-target DNA (firefly luciferase vector) and 0.1 μg PRL-TK (TK-driven Renilla luciferase expression vector) with lipofectamine 2000. Following transfection (48 h), luciferase activities were measured with a dual luciferase reporter assay kit (Promega) on a luminometer (Lumat LB9507)³¹. For all experiments, transfection took place 24 h after starvation of cells in serum-free medium.

Data Analysis

Group data are expressed as mean±S.E. Statistical comparisons (performed using ANOVA followed by Dunnett's method) were carried out using Microsoft Excel. A two-tailed p<0.05 was taken to indicate a statistically significant difference. Nonlinear least square curve fitting was performed with CLAMPFIT in pCLAMP 8.0 or GraphPad Prism.

Although the present invention has been described hereinabove by way of preferred embodiments thereof, it can be modified, without departing from the spirit and nature of the subject invention as defined in the appended claims.

### REFERENCES

1. Meister, G. & Tuschl, T. Mechanisms of gene silencing by double-stranded RNA. Nature 431, 343-349 (2004).
2. Alvarez-Garcia, I. & Miska, E. A. MicroRNA functions in animal development and human disease. Development 132, 4653-4662 (2005).
3. Miranda, K. C. et al. A pattern-based method for the identification of MicroRNA binding sites and their corresponding heteroduplexes. Cell 126, 1203-1217 (2006).
4. Rao, P. K., Kumar, R. M., Farkhondeh, M., Baskerville, S. & Lodish, H. F. Myogenic factors that regulate expression of muscle-specific microRNAs. Proc. Natl. Acad. Sci. U.S.A. 103, 8721-8726 (2006).
5. Zhao, Y., Samal, E. & Srivastava, D. Serum response factor regulates a muscle-specific microRNA that targets Hand2 during cardiogenesis. Nature 436, 214-220 (2005).
6. Chen, J. F. et al. The role of microRNA-1 and microRNA-133 in skeletal muscle proliferation and differentiation. Nat. Genet. 38, 228-233 (2006).
7. Clements-Jewery, H., Hearse, D. J. & Curtis, M. J. Phase 2 ventricular arrhythmias in acute myocardial infarction: a neglected target for therapeutic antiarrhythmic drug development and for safety pharmacology evaluation. Br. J. Pharmacol. 145, 551-564 (2005).
8. Ghuran, A. V. & Camm, A. J. Ischaemic heart disease presenting as arrhythmias. Br. Med. Bull. 59, 193-210 (2001).
9. Krutzfeldt, J. et al. Silencing of microRNAs in vivo with ‘antagomirs’. Nature 438, 685-689 (2005).
10. Cheng, A. M., Byrom, M. W., Shelton, J. & Ford, L. P. Antisense inhibition of human miRNAs and indications for an involvement of miRNA in cell growth and apoptosis. Nucleic Acids Res. 33, 1290-1297 (2005).
11. Kitsis, R. N., Buttrick, P. M., McNally, E. M., Kaplan, M. L. & Leinwand, L. A. Hormonal modulation of a gene injected into rat heart in vivo. Proc. Natl. Acad. Sci. U.S.A. 88, 4138-4142 (1991).
12. Rajan, S., Radhakrishnan, J. & Rajamanickam, C. Direct injection and expression in vivo of full-length cDNA of the cardiac isoform of alpha-2 macroglobulin induces cardiac hypertrophy in the rat heart. Basic Res. Cardiol. 98, 39-49 (2003).
13. Sarkar, N. et al. Nonsurgical direct delivery of plasmid DNA into rat heart: time course, dose response, and the influence of different promoters on gene expression. J. Cardiovasc. Pharmacol. 39, 215-224 (2002).
14. Jongsma, H. J. & Wilders, R. Gap junctions in cardiovascular disease. Circ. Res. 86, 1193-1197 (2000).
15. Diaz, R. J. et al. Selective inhibition of inward rectifier K+ channels (Kir2.1 or Kir2.2) abolishes protection by ischemic preconditioning in rabbit ventricular cardiomyocytes. Circ. Res. 95, 325-332 (2004).
16. Yue, P. et al. Ischemia impairs the association between connexin 43 and M3 subtype of acetylcholine muscarinic receptor (M3-mAChR) in ventricular myocytes. Cell Physiol Biochem. 17, 129-136 (2006).
17. Lerner, D. L., Yamada, K. A., Schuessler, R. B. & Saftz, J. E. Accelerated onset and increased incidence of ventricular arrhythmias induced by ischemia in Cx43-deficient mice. Circulation 101, 547-552 (2000).
18. Guerrero, P. A. et al. Slow ventricular conduction in mice heterozygous for a connexin43 null mutation. J. Clin. Invest. 99, 1991-1998 (1997).
19. McLerie, M. & Lopatin, A. N. Dominant-negative suppression of IK1 in the mouse heart leads to altered cardiac excitability. J. Mol. Cell. Cardiol. 35, 367-378 (2003).
20. Sayed, D., Hong, C., Chen, I-Y., Lypowy, J., Abdellatif, M. MicroRNAs play an essential role in the development of cardiac hypertrophy. Circ. Res. 100, 416-424 (2007).
21. Van Rooij, E. et al. A signature pattern of stress-responsive microRNAs that can evoke cardiac hypertrophy and heart failure. Proc. Natl. Acad. Sci. U.S.A. 103, 18255-18260 (2006).
22. Yang, B. et al. Choline produces cytoprotective effects against ischemic myocardial injuries: evidence for the role of cardiac M3 subtype muscarinic acetylcholine receptors. Cell. Physiol. Biochem. 16, 163-174 (2005).
23. Curtis, M. J. & Walker, M. J. Quantification of arrhythmias using scoring systems: an examination of seven scores in an in vivo model of regional myocardial ischemia. Cardiovasc. Res. 22, 656-665 (1988).
24. Salameh, A. et al. Chronic regulation of the expression of gap junction proteins connexin40, connexin43, and connexin45 in neonatal rat cardiomyocytes. Eur. J. Pharmacol. 503, 9-16 (2004).
25. Yang, B. et al. Choline produces cytoprotective effects against ischemic myocardial injuries: evidence for the role of cardiac M₃ subtype muscarinic acetylcholine receptors. Cell. Physiol. Biochem. 16, 163-174 (2005).
26. Shao, Q., Wang, H., McLachlan, E., Veitch, G. I. & Laird, D. W. Down-regulation of Cx43 by retroviral delivery of small interfering RNA promotes an aggressive breast cancer cell phenotype. Can. Res. 65, 2705-2711 (2005).
27. Sanchez-Alvarez, R., Paino, T., Herrero-Gonzalez, S., Medina, J. M. & Tabernero, A. Tolbutamide reduces glioma cell proliferation by increasing connexin43, which promotes the up-regulation of p21 and p27 and subsequent changes in retinoblastoma phosphorylation. Glia 54, 125-134 (2006). Rinne, A., Littwitz, C., Kienitz, M. C., Gmerek, A., Bosche, L. I., Pott, L & Bender, K. Gene silencing in adult rat cardiac myocytes in vitro by adenovirus-mediated RNA interference. J. Muscle Res. Cell Motil. 27, 413-421 (2006).
28. Kitsis, R. N., Buttrick. P. M., McNally, E. M., Kaplan, M. L. & Leinwand. L. A. Hormonal modulation of a gene injected into rat heart in vivo. Proc. Natl. Acad. Sci. U.S.A. 88, 4138-4142 (1991).
29. Rajan, S., Radhakrishnan, J. & Rajamanickam, C. Direct injection and expression in vivo of full-length cDNA of the cardiac isoform of alpha-2 macroglobulin induces cardiac hypertrophy in the rat heart. Basic Res. Cardiol. 98, 39-49 (2003).
30. Pang, L., Koren, G., Wang, Z. & Nattel S. Tissue-specific expression of two human Caᵥ1.2 isoforms under the control of distinct 5′-flanking regulatory elements. FEBS Lett. 546, 349-354 (2003).
31. Curtis, M. J. & Walker, M. J. Quantification of arrhythmias using scoring systems: an examination of seven scores in an in vivo model of regional myocardial ischemia. Cardiovasc. Res. 22, 656-65 (1988).
32. Guerrero, P. A. et al. Slow ventricular conduction in mice heterozygous for a connexin43 null mutation. J. Clin. Invest. 99, 1991-1998 (1997).
33. Yue, P. et al. Ischemia impairs the association between connexin 43 and M3 subtype of acetylcholine muscarinic receptor (M3-mAChR) in ventricular myocytes. Cell. Physiol. Biochem. 17, 129-136 (2006).
34. Zhang, Y. et al. Restoring depressed HERG K⁺ channel function as a mechanism for insulin treatment of the abnormal QT prolongation and the associated arrhythmias in diabetic rabbits. Am. J. Physiol. 291, H1446-H1455 (2006).
35. Wang, Z., Yue, L., White, M., Pelletie, r G. & Naftel, S. Differential expression of inward rectifier potassium channel mRNA in human atrium versus ventricle and in normal versus failing hearts. Circulation 98, 2422-2428 (1998).
36. Wang, H. et al. HERG K⁺ Channel, a regulator of tumor cell apoptosis and proliferation. Can. Res. 62, 4843-4848 (2002).
37. Griffiths-Jones, S, Nucleic Acids Res. 32, D109-D111 (2004).
38. Salameh, A., Schneider, P., Muhlberg, K., Hagendorff, A., Dhein, S. & Pfeiffer, D. Chronic regulation of the expression of gap junction proteins connexin40, connexin43, and connexin45 in neonatal rat cardiomyocytes. Eur. J. Pharmacol. 503, 9-16 (2004).

TABLE 1
Characteristics of patients from whom the human ventricular preparations
were obtained for use in the present study

| | HH | CAD1 | CAD2 | CAD3 | CAD4 | CAD5 | CAD6 | CAD7 |
|---|---|---|---|---|---|---|---|---|
| Sex (M/F) | 3 M & | F | M | F | M | M | F | M |
| | 3 F | | | | | | | |
| Age (year) | 55.3 | 65 | 45 | 46 | 66 | 64 | 58 | 51 |
| | (mean) | | | | | | | |
| CAD Diagnosed by: | | | | | | | | |
| ECG & | | + | + | + | + | + | + | + |
| Stress test | | | | | | | | |
| Cardiac | | + | | + | + | | | |
| catheterization | | | | | | | | |
| Coronary | | | + | | + | | | |
| angiography | | | | | | | | |
| MRI | | | | + | | + | | |
| Others disease | 2 CAN | DM | HT | CAN | | DM | CAN | CAN |

CAD: coronary artery disease;
CAD1-CAD7: patients with CAD labeled from 1 to 7 for easy identification;
CAN: cancer;
DM: diabetes mellitus;
HH: healthy hearts (no diagnosed heart disease);
HT: hypertension;
MRI: magnetic resonance imaging.

TABLE 2
Changes of QRS duration and QT interval in rat hearts with intramuscular
injection of various constructs

| | | |
|---|---|---|
| QRS Duration | QT Interval | Heart Rate |
| (ms) | (ms) | (beat/min) |

Healthy Hearts (HH)

| | | | |
|---|---|---|---|
| Control | 18 ± 1 | 37 ± 2 | 350 ± 18 |
| (n = 25) | | | |
| WT miR-1 | 25 ± 2* | 45 ± 3* | 362 ± 14 |
| (n = 20) | | | |
| MT miR-1 | 19 ± 1 | 39 ± 3 | 355 ± 15 |
| (n = 20) | | | |
| AMO-1 | 16 ± 1 | 34 ± 2 | 348 ± 14 |
| (n = 20) | | | |
| WT miR-1 + AMO-1 | 20 ± 2† | 40 ± 3† | 352 ± 17 |
| (n = 20) | | | |
| WT miR-1 + Mis-AMO-1 | 26 ± 2* | 43 ± 3* | 365 ± 32 |
| (n = 10) | | | |

Myocardial Infarction (MI)

| | | | |
|---|---|---|---|
| Control | 27 ± 2 | 43 ± 3 | 369 ± 24 |
| (n = 20) | | | |
| WT miR-1 | 35 ± 2* | 55 ± 4* | 371 ± 27 |
| (n = 14) | | | |
| MT miR-1 | 25 ± 2 | 44 ± 3 | 365 ± 18 |
| (n = 14) | | | |
| AMO-1 | 19 ± 2* | 38 ± 3* | 363 ± 19 |
| (n = 14) | | | |
| WT miR-1 + AMO-1 | 21 ± 2† | 40 ± 4† | 366 ± 11 |
| (n = 14) | | | |
| WT miR-1 + Mis-AMO-1 | 33 ± 3* | 53 ± 3* | 368 ± 35 |
| (n = 10) | | | |

*p < 0.05 vs. control and
†p <0.05 vs. WT miR-1 alone; unpaired Student t-test.

What is claimed is:

1. A method of preventing or treating a cardiac condition in a mammal, the method comprising administering a therapeutically effective amount of an inhibitor of miR-1 to said mammal.

2. The method of claim 1, wherein the cardiac condition is selected from the group consisting of cardiac arrhythmia, myocardial infarction, myocardial ischemia, angina, and coronary artery disease.

3. The method of claim 2, wherein the arrhythmia is a widening of the QRS complex and a prolonged QT interval or a phase II arrhythmia.

4. The method of claim 1, wherein the inhibitor of miR-1 is a 2′-O-methyl-modified antisense oligoribonucleotide (AMO) specific to miR-1.

5. The method of claim 1, wherein miR-1 has the nucleic acid sequence set forth in SEQ ID NO:1.

6. The method of claim 4, wherein the inhibitor of miR-1 is the exact antisense of the mature miR-1 mRNA sequence.

7. The method of claim 4, wherein the AMO has the nucleic acid sequence set forth in SEQ ID NO:13.

8. The method of claim 1, wherein the inhibitor is delivered prior to the onset of the cardiac condition.

9. The method of claim 1, wherein the inhibitor is delivered after the onset of the cardiac condition.

10. The method of claim 1, wherein the mammal is a human.

11. A method of diagnosing a cardiac condition in a mammal, the method comprising measuring the expression level of miR-1 in the mammal and comparing the expression level to a standard miR-1 level, wherein an increase in miR-1 expression level compared to the standard level in the mammal indicates the mammal has a cardiac condition.

12. The method of claim 11, wherein an increase of approximately 2.8-fold or more of miR-1 level indicates that the mammal has a cardiac condition.

13. The method of claim 11, wherein measuring the expression level of miR-1 comprises performing a reverse transcription polymerase chain reaction of miR-1 using a total RNA sample from the mammal.

14. The method claim 11, wherein the cardiac condition is selected from the group consisting of cardiac arrhythmia, myocardial infarction, myocardial ischemia, angina, and coronary artery disease.

15. The method of claim 11, wherein the arrhythmia is a widening of the QRS complex and a prolonged QT interval or phase II arrhythmia.

16. The method of claim 11, wherein miR-1 has the nucleic acid sequence set forth in SEQ ID NO:1.

17. The method of claim 11, wherein the mammal is a human.

18. The method of claim 11, wherein the method of diagnosing is performed prior to the onset of any overt symptoms of the cardiac conditions occurring.

19. The method of claim 11, wherein the method of diagnosing is performed after the onset of any overt symptoms of the cardiac condition occurring.

20. A method of inducing a cardiac condition in a mammal, the method comprising administering miR-1 to the cardiovascular system of the mammal in an amount effective to induce a cardiac condition.

21. The method of claim 20, wherein the mammal is a rat.

22. The method of claim 21, wherein the miR-1 has the nucleic acid sequence set forth in SEQ ID NO:2.

23. A method of preventing or treating a cardiac condition in a mammal, the method comprising administering a therapeutically effective amount of a compound that increases the expression of GJAI and KCNJ2.

24. An isolated nucleic acid, said nucleic acid having the sequence set forth in SEQ ID NOS:12 or 13.


SEQUENCE LISTING (23 sequences)

SEQ ID NO: 1 - RNA, 22 nt, Artificial Sequence
  Description of Artificial Sequence Synthetic oligonucleotide
uggaauguaaagaaguauguaa

SEQ ID NO: 2 - RNA, 22 nt, Artificial Sequence
  Description of Artificial Sequence Synthetic oligonucleotide
uggaauguaaagaaguguguau

SEQ ID NO: 3 - RNA, 22 nt, Artificial Sequence
  Description of Artificial Sequence Synthetic oligonucleotide
uggaauguaaagaaguauguau

SEQ ID NO: 4 - RNA, 23 nt, Artificial Sequence
  Description of Artificial Sequence Synthetic oligonucleotide
uuacuaauuuguuugacauucca

SEQ ID NO: 5 - RNA, 23 nt, Artificial Sequence
  Description of Artificial Sequence Synthetic oligonucleotide
aaacuaauguguuugacauucca

SEQ ID NO: 6 - RNA, 23 nt, Artificial Sequence
  Description of Artificial Sequence Synthetic oligonucleotide
ccccccaaaaaaaaaacauucca

SEQ ID NO: 7 - RNA, 23 nt, Artificial Sequence
  Description of Artificial Sequence Synthetic oligonucleotide
aaacuaauuuguuugacauucca

SEQ ID NO: 8 - RNA, 22 nt, Artificial Sequence
  Description of Artificial Sequence Synthetic oligonucleotide
auccccgcuaaaaaacauucca

SEQ ID NO: 9 - RNA, 22 nt, Artificial Sequence
  Description of Artificial Sequence Synthetic oligonucleotide
gcuuuuccuuuugcacauucca

SEQ ID NO: 10 - RNA, 24 nt, Artificial Sequence
  Description of Artificial Sequence Synthetic oligonucleotide
gcuuuucuuucuuugcacauucca

SEQ ID NO: 11 - RNA, 24 nt, Artificial Sequence
  Description of Artificial Sequence Synthetic oligonucleotide
gcuuuucucucuuagcacauucca

SEQ ID NO: 12 - DNA, 22 bp, Artificial Sequence
  Description of Artificial Sequence Synthetic oligonucleotide
atacacacttctttacattcca

SEQ ID NO: 13 - DNA, 22 bp, Artificial Sequence
  Description of Artificial Sequence Synthetic oligonucleotide
ttacatacttctttacattcca

SEQ ID NO: 14 - DNA, 22 bp, Artificial Sequence
  Description of Artificial Sequence Synthetic oligonucleotide
cgctacacttctttatcggtta

SEQ ID NO: 15 - RNA, 22 nt, Artificial Sequence
  Description of Artificial Sequence Synthetic oligonucleotide
guagccaaaaagaaguguguau

SEQ ID NO: 16 - RNA, 23 nt, Artificial Sequence
  Description of Artificial Sequence Synthetic oligonucleotide
aaacuaauguguuuguuggcuac

SEQ ID NO: 17 - RNA, 23 nt, Artificial Sequence
  Description of Artificial Sequence Synthetic oligonucleotide
ccccccaaaaaaaaauuggcuac

SEQ ID NO: 18 - RNA, 24 nt, Artificial Sequence
  Description of Artificial Sequence Synthetic oligonucleotide
gcuuuucuuucuuugcuuggcuac

SEQ ID NO: 19 - RNA, 22 nt, Artificial Sequence
  Description of Artificial Sequence Synthetic oligonucleotide
guagccaaaaagaaguauguaa

SEQ ID NO: 20 - RNA, 23 nt, Artificial Sequence
  Description of Artificial Sequence Synthetic oligonucleotide
uuacuaauuuguuuguuggcuac

SEQ ID NO: 21 - RNA, 22 nt, Artificial Sequence
  Description of Artificial Sequence Synthetic oligonucleotide
gcuuuuccuuuugcuuggcuac

SEQ ID NO: 22 - DNA, 19 bp, Artificial Sequence
  Description of Artificial Sequence Synthetic oligonucleotide
gaagttcaagtacgggatt

SEQ ID NO: 23 - DNA, 19 bp, Artificial Sequence
  Description of Artificial Sequence Synthetic oligonucleotide
ggtgtgttacagacgagtg